(19) United States
(12) Patent Application Publication
(10) Pub. No.: US 2010/0285455 A1
(43) Pub. Date: Nov. 11, 2010

(54) PREDICTION OF SCHIZOPHRENIA RISK USING HOMOZYGOUS GENETIC MARKERS

(75) Inventors: Todd Lencz, New York, NY (US); Anil K. Malhotra, Bye Brook, NY (US); John m. Kane, Long Island City, NY (US)

Correspondence Address: AMSTER, ROTHSTEIN & EBENSTEIN LLP, 90 PARK AVENUE, NEW YORK, NY, 10016, US

(73) Assignee: THE FEINSTEIN INSTITUTE MEDICAL RESEARCH, Manhasset, NY (US)

(21) Appl. No.: 12/452,097

(22) PCT Filed: Jun. 10, 2008
(86) PCT No.: PCT/US2008/007247

§ 371 Date: Jul. 21, 2010

Related U.S. Application Data
(60) Provisional application No. 60/934,728, filed on Jun. 15, 2007.

(51) Int. Cl. C12Q 1/68

(52) U.S. Cl. 435/6

(57) ABSTRACT

Provided are methods of identifying a genetic profile influencing the relative probability of a subject manifesting a phenotype that is at least partially heritable. Also provided are methods of determining the relative likelihood that a subject will manifest a phenotype that is at least partially heritable. Additionally, methods of determining the relative risk of a human subject for manifesting schizophrenia are provided. Further provided are methods of screening a human embryo in vitro for the risk of becoming a human manifesting schizophrenia. Also, methods of identifying a single nucleotide polymorphism (SNP) variant affecting the risk of a human subject for manifesting schizophrenia are provided. Methods of screening for a compound that may affect schizophrenia are additionally provided.

### STATEMENT REGARDING FEDERALLY SPONSORED RESEARCH OR DEVELOPMENT

This application claims the benefit of U.S. Provisional Patent Application No. 60/934,728 filed on Jun. 15, 2007, the contents of which are hereby incorporated by reference.

### STATEMENT REGARDING FEDERALLY SPONSORED RESEARCH OR DEVELOPMENT

This invention was supported by NIH grants MH065580, MH074543, and MH001760. As such, the U.S. Government has certain rights in this invention.

### BACKGROUND OF THE INVENTION

(1) Field of the Invention

The present invention generally relates to prediction of disease risk. More specifically, the invention is directed to methods of identifying a disease risk genotype. The invention is also directed to methods for determining the relative risk of manifesting schizophrenia.

(2) Description of the Related Art

The recent development of microarray platforms, capable of genotyping hundreds of thousands of single nucleotide polymorphisms (SNPs), has provided an opportunity to rapidly identify novel susceptibility genes for complex phenotypes. Studies employing genotyping microarrays have typically utilized a whole genome association (WGA) approach, in which each SNP is examined individually for association with disease (Hirschhorn and Daly, 2005); multiple testing requires that statistical thresholds for WGA approach 10⁻⁷ or lower (Carlson et al., 2004). Given the presumably polygenic nature of complex illness, this conservative strategy inevitably results in false negatives in the search for susceptibility genes (Storey and Tibshirani, 2003).

Schizophrenia (SCZ) is a disease with estimated lifetime morbid risk approaching 1% worldwide. Although genetic epidemiologic studies have revealed high heritability estimates (70-80%) for SCZ, identification of susceptibility genes remains challenging. As with other complex diseases, linkage studies have revealed multiple candidate regions with modest LOD scores (Lewis et al., 2003), while studies of individual candidate genes are inherently limited in scope.

In light of the above, improved methods for identifying disease (especially SCZ) susceptibility loci are needed. The present invention addresses that need.

### SUMMARY OF THE INVENTION

The inventors have developed a method for identifying genetic loci influencing a heritable phenotype. The method utilizes the identification of long runs of consecutive SNP loci that are homozygous, where these “runs of homozygosity” (ROH) are associated with the occurrence of the phenotype. This invention was validated by identifying ROH associated with schizophrenia.

The present invention is directed to methods of identifying a genetic profile influencing the relative probability of a subject manifesting a phenotype that is at least partially heritable. The methods comprise obtaining a genomic DNA sample from each individual in two populations of individuals, the first population consisting of individuals manifesting the phenotype and the second population consisting of individuals not manifesting the phenotype; and analyzing the genomic DNA from each individual in the first population and the second population to identify a run of homozygosity (ROH) present in the first population more often, or less often, than in the second population. An ROH present in the first population more often than in the second population indicates that the presence of the ROH is a genetic profile associated with increased probability for manifesting the phenotype, and an ROH present in the first population less often than in the second population indicates that the presence of the ROH is a genetic profile associated with decreased probability for manifesting the phenotype. With these methods, an ROH is a series of consecutive known single nucleotide polymorphism (SNP) positions that are homozygous in the genome of an individual.

The invention is also directed to methods of determining the relative likelihood that a subject will manifest a phenotype. The methods comprise determining whether the subject has a genetic profile associated with an increased likelihood for manifesting the phenotype. The genetic profile is identified by the method described above. In these methods, a subject having the genetic profile has an increased likelihood of manifesting the phenotype over a subject not having the genetic profile.

Additionally, the invention is directed to methods of determining the relative risk of a human subject for manifesting schizophrenia. The methods comprise determining the presence of a first run of homozygosity (ROH) in the genome of the subject, where the presence of the first ROH indicates the subject has an increased risk for manifesting schizophrenia over a subject not having the first ROH. In these methods, the first ROH is a series of consecutive single nucleotide polymorphism (SNP) positions that are homozygous in the subject from one of roh250, roh321, roh314, roh52, roh15, roh129, roh291, roh55, or roh173 as defined in Table 2.

The invention is further directed to other methods of determining the relative risk of a human subject for manifesting schizophrenia. The methods comprise determining whether the subject has a run of homozygosity (ROH) that contains at least 80% of the SNPs in at least one of the three locations identified in Supplementary Table 2 as correlated with schizophrenia. A subject having an ROH that contains at least 80% of the SNPs in at least one of the three locations identified in Supplementary Table 2 has an increased risk for manifesting schizophrenia over a subject not having such an ROH.

Also, the invention is directed to methods of screening a human embryo in vitro for the risk of becoming a human manifesting schizophrenia. The methods comprise determining the presence of a first run of homozygosity (ROH) in the genome of the embryo, where the presence of the first ROH indicates the embryo has an increased risk for manifesting schizophrenia over an embryo not having the first ROH. Here, the first ROH is a series of consecutive single nucleotide polymorphism (SNP) positions that are homozygous in the subject from one of roh250, roh321, roh314, roh52, roh15, roh129, roh291, roh55, or roh173 as defined in Table 2.

The invention is additionally directed to methods of identifying a single nucleotide polymorphism (SNP) variant affecting the risk of a human subject for manifesting schizophrenia. The methods comprise identifying a run of homozygosity (ROH) present more often in a first population of individuals having schizophrenia than in a second population of individuals not having schizophrenia, then identifying a single nucleotide polymorphism (SNP) within the ROH or within 500 kB of the ROH, where a first variant of the SNP is present in the first population more often than in the second population. In these methods, the presence of the first variant of the SNP in a subject indicates that the subject has a greater risk for manifesting schizophrenia than the absence of the first variant. Here, an ROH is a series of consecutive known SNP positions that are homozygous in the genome of an individual.

The invention is also directed to additional methods of determining the relative risk of a human subject for manifesting schizophrenia. The methods comprise determining whether the subject has a SNP genotype associated with schizophrenia as identified by the method described immediately above. A subject with the SNP genotype has an increased risk for manifesting schizophrenia over a subject with a different genotype.

Further, the invention is directed to methods of screening for a compound that may affect schizophrenia. The methods comprise determining whether the compound affects expression or activity of a gene selected from the group consisting of DYNC2H1, CRHR1, IMP5, MAPT, STH, KIAA1267, LRRC37A, ARL17, LRRC37A2, WNTT3, WNT9B, GOSR2, RPRML, CDC27, CHN1, ATP5GS3, DUSP12, ATF6, OLFML2B, SGCD, MRPL22, GPHN, C14orf54, MPP5, ATP6V1D, EIF2S1, PLEK2, GULP1, DIRC1, COL3A1, COL5A2, WDR75, SLC40A1, NS3TP1, ASNSD1, ANKAR, OSGEPL1, ORMDL1, PMS1, GDF8, IMPAD1, SNTG1 and SORCS1. Here, a compound that affects expression or activity of the gene may affect schizophrenia.

### BRIEF DESCRIPTION OF THE DRAWING

FIG. 1 is a graphical depiction of statistical comparisons (SCZ vs. control) at individual SNPs within roh172 on Chromosome 8q. Chromosomal context is depicted in ideogram at top. Gene location (Build 35 coordinates) for SNTG1 is depicted immediately below ideogram. Coding region of SNTG1 is indicated by red dotted line; exons are indicated by horizontal lines. Gray box depicts −log₁₀ P-values for case-control comparisons at each binarized SNP.

### DETAILED DESCRIPTION OF THE INVENTION

The inventors have developed a method for identifying genetic loci influencing a heritable phenotype. The method utilizes the identification of long runs of consecutive SNP loci that are homozygous, where these “runs of homozygosity” (ROH) are associated with the occurrence of the phenotype. This invention was validated by identifying ROH associated with schizophrenia. See Example.

The present invention is directed to methods of identifying a genetic profile influencing the relative probability of a subject manifesting a phenotype that is at least partially heritable. The methods comprise obtaining a genomic DNA sample from each individual in two populations of individuals, the first population consisting of individuals manifesting the phenotype and the second population consisting of individuals not manifesting the phenotype; and analyzing the genomic DNA from each individual in the first population and the second population to identify a run of homozygosity (ROH) present in the first population more often, or less often, than in the second population. An ROH present in the first population more often than in the second population indicates that the presence of the ROH is a genetic profile associated with increased probability for manifesting the phenotype, and an ROH present in the first population less often than in the second population indicates that the presence of the ROH is a genetic profile associated with decreased probability for manifesting the phenotype. With these methods, an ROH is a series of consecutive known single nucleotide polymorphism (SNP) positions that are homozygous in the genome of an individual.

The “consecutive known SNP positions” that are interrogated to identify an ROH are consecutive SNP positions that are chosen as part of the method; this is not meant to necessarily include every consecutive SNP known in the genome region that is being interrogated. For example, the Example describes usefully applying the invention method by using an Affymetrix gene chip that has a mean spacing of 5.8 kB between SNPs. The skilled artisan could identify a useful collection of SNPs without undue experimentation for any particular application of the method.

The ROH in these methods should cover a long enough stretch of the genome, and include a sufficient number of SNP positions, to provide adequate assurance that the ROH reflects a true difference between the two populations. Preferably, the ROH is at least 50 kB in length. More preferably, the ROH is at least 100 kB in length. Even more preferably, the ROH is at least 200 kB in length. Most preferably, the ROH is at least 500 kB in length.

The SNPs in the ROH should also occur at sufficient density such that there is a reasonable assurance that the presence of the consecutive homozygous SNP positions adequately reflects the true occurrence of predominantly homozygous SNPs that are not interrogated in the ROH. Preferably, the consecutive known SNP positions are an average of less than 50 kB apart. More preferably, the consecutive known SNP positions are an average of less than 20 kB apart. Even more preferably, the consecutive known SNP positions are an average of less than 10 kB apart. Most preferably, the consecutive known SNP positions are an average of less than 5 kB apart.

The density of the SNP positions and the length of the ROH determines the number of SNP positions covered by the ROH. Preferably, the ROH is a series of at least 10 consecutive known SNP positions that are homozygous. More preferably, the ROH is a series of at least 20 consecutive known SNP positions that are homozygous. Even more preferably, the ROH is a series of at least 50 consecutive known SNP positions that are homozygous. Most preferably, the ROH is a series of at least 100 consecutive known SNP positions that are homozygous.

The “subject” for these methods can be any mammal, including a fetus or embryo. The subject is preferably a human.

It is to be understood that the region surrounding the identified ROH (e.g., within 1000 kB on each side of the ROH, preferably 500 kB, more preferably 200 kB, even more preferably 100 kB) is tightly linked to the ROH such that the ROH could potentially be identified by identifying the genotype at a SNP position, or a series of SNP positions (e.g., consecutive positions) within those regions. Thus, the present methods encompass the identification of the ROH by evaluating the genotype of regions surrounding the identified ROH.

The ROHs identified as above that are associated with the phenotype are also useful for identifying the SNPs that are at least partially responsible for the association of the ROH with the phenotype. Such an identification can lead to more precise and easier methods of estimating the relative probability that the subject will manifest the phenotype. Additionally, the association of the SNP with a genetic change in a gene could be useful for further understanding the phenotype.

Thus, in some aspects, these methods further comprise identifying all SNPs having a genotype that occurs with a different frequency in the first population than in the second population, then identifying any runs of SNPs with such differences extending at least 50 consecutive SNPs in length. In these aspects, a subject having such a run of SNPs identical with the run in the first population has an increased probability for manifesting the phenotype.

The phenotype can be any trait having polygenic inheritance, including but not limited to characteristics relating to the development, anatomy, biochemistry or physiology of a tissue, organ or cell type, including but not limited to: therapeutic responses including responses to drugs, intelligence, muscle mass, presence and characteristics of immune cells, ability to produce milk, or leanness of meat. It is to be understood that these methods can also be used to evaluate the likely quantitative degree that a phenotype will manifest itself in the subject.

Preferably, the disease or condition is a disease. Nonlimiting examples include Parkinson's disease, Alzheimer's disease, a cancer, a cardiovascular disease, an infectious disease, an autoimmune disease, and type 2 diabetes. The disease can also be a psychiatric disease. Nonlimiting examples include schizophrenia, bipolar disorder, depression, or autism. The analysis can also potentially encompass evaluation of the likelihood of achieving a particular level of severity of a disease, or rapidity of disease development.

The genetic profiles identified by the above methods can be used to determine the likelihood that a subject with manifest the phenotype. The invention is thus also directed to methods of determining the relative likelihood that a subject will manifest a phenotype. The methods comprise determining whether the subject has a genetic profile associated with an increased likelihood for manifesting the phenotype. The genetic profile is identified by the method described above. In these methods, a subject having the genetic profile has an increased likelihood of manifesting the phenotype over a subject not having the genetic profile.

As discussed above, the subject being evaluated in these methods can be an adult animal or an embryo or fetus, including a human embryo or fetus, e.g., by analysis of amniotic fluid, chorionic villi. In some aspects, the subject is an embryo, in others the subject is a fetus. These methods can also be used in breeding farm or companion animals.

Preferably, the phenotype is a disease. Nonlimiting examples include Parkinson's disease, Alzheimer's disease, a cancer, a cardiovascular disease, an infectious disease, an autoimmune disease, and type 2 diabetes. The disease can also be a psychiatric disease. Nonlimiting examples include schizophrenia, bipolar disorder, depression, or autism. The analysis can also potentially encompass evaluation of the likelihood of the subject achieving a particular level of severity of a disease, or rapidity of disease development.

As discussed above and in the Example, the genetic profiling method described above was used to identify nine ROHs associated with schizophrenia. These ROHs are useful for evaluating the relative risk for a human subject manifesting schizophrenia.

Thus, the invention is additionally directed to methods of determining the relative risk of a human subject for manifesting schizophrenia. The methods comprise determining the presence of a first run of homozygosity (ROH) in the genome of the subject, where the presence of the first ROH indicates the subject has an increased risk for manifesting schizophrenia over a subject not having the first ROH. In these methods, the first ROH is a series of consecutive single nucleotide polymorphism (SNP) positions that are homozygous in the subject from one of roh250, roh321, roh314, roh52, roh15, roh129, roh291, roh55, or roh173 as defined in Table 2.

Preferably, the first ROH is a series of at least 50 consecutive homozygous SNP positions. More preferably, the first ROH is a series 100 consecutive homozygous SNP positions. Most preferably, the first ROH is all of the SNP positions that are homozygous in the subject from roh250, roh321, roh314, roh52, roh15, roh129, roh291, roh55, or roh173.

Preferably, the subject is evaluated for the presence of more than one ROH. Thus, the methods preferably further comprise determining the presence of a second ROH in the genome of the subject, where the second ROH is from one of roh250, roh321, roh314, roh52, roh15, roh129, roh291, roh55, or roh173 that is different from the first ROH. Here, the presence of the second ROH indicates the subject has an increased risk for manifesting schizophrenia over a subject not having the second ROH. It is preferred that the presence of roh250 is determined, since that ROH was the most strongly associated with schizophrenia.

Most preferably, the subject is evaluated for the presence of all of the ROHs. Thus, preferably, wherein positions in the genome of the subject corresponding to each of roh250, roh321, roh314, roh52, roh15, roh129, roh291, roh55, and roh173 are evaluated for the consecutive homozygous SNP positions, wherein an increasing number of ROHs present in the subject indicates an increasing risk in the subject for manifesting schizophrenia.

The subject in these methods can be a human adult, child, infant, fetus or embryo. In some aspects, the subject is an embryo. In others, the subject is a fetus.

Further evaluations, as discussed in the example, led to the identification of three additional ROHs associated with schizophrenia, described in Supplementary Table 2. The invention is thus further directed to additional methods of determining the relative risk of a human subject for manifesting schizophrenia. The methods comprise determining whether the subject has a run of homozygosity (ROH) that contains at least 80% of the SNPs in at least one of the three locations identified in Supplementary Table 2 as correlated with schizophrenia. In these methods, a subject having an ROH that contains at least 80% of the SNPs in at least one of the three locations identified in Supplementary Table 2 has an increased risk for manifesting schizophrenia over a subject not having such an ROH.

Preferably, these methods comprise determining whether the subject has an ROH that contains at least 90% of the SNPs in at least one of the three locations identified in Supplementary Table 2 as correlated with schizophrenia, wherein a subject having an ROH that contains at least 90% of the SNPs in at least one of the three locations identified in Supplementary Table 2 has an increased risk for manifesting schizophrenia over a subject not having such an ROH. Most preferably, the methods comprise determining whether the subject has an ROH that contains 100% of the SNPs in at least one of the three locations identified in Supplementary Table 2 as correlated with schizophrenia, wherein a subject having an ROH that contains 100% of the SNPs in at least one of the three locations identified in Supplementary Table 2 has an increased risk for manifesting schizophrenia over a subject not having such an ROH.

These methods can be applied to analysis of human embryos. Thus, the invention is additionally directed to methods of screening a human embryo in vitro for the risk of becoming a human manifesting schizophrenia. The methods comprise determining the presence of a first run of homozygosity (ROH) in the genome of the embryo, where the presence of the first ROH indicates the embryo has an increased risk for manifesting schizophrenia over an embryo not having the first ROH. In these methods, the first ROH is a series of consecutive single nucleotide polymorphism (SNP) positions that are homozygous in the subject from one of roh250, roh321, roh314, roh52, roh15, roh129, roh291, roh55, or roh173 as defined in Table 2.

The individual SNPs in the ROH can be further evaluated for association with schizophrenia. The invention is thus further directed to methods of identifying a single nucleotide polymorphism (SNP) variant affecting the risk of a human subject for manifesting schizophrenia. The methods comprise identifying a run of homozygosity (ROH) present more often in a first population of individuals having schizophrenia than in a second population of individuals not having schizophrenia, then identifying a single nucleotide polymorphism (SNP) within the ROH, or within 500 kB of the ROH, where a first variant of the SNP is present in the first population more often than in the second population, where the presence of the first variant of the SNP in a subject indicates that the subject has a greater risk for manifesting schizophrenia than the absence of the first variant, Here, an ROH is a series of at least 50 consecutive known SNP positions that are homozygous in the genome of an individual.

The SNP variant(s) identified from the ROHs can be used to determine the relative risk of schizophrenia. Thus, the invention is directed to additional methods of determining the relative risk of a human subject for manifesting schizophrenia. The methods comprise determining whether the subject has a SNP genotype associated with schizophrenia as identified by the method described immediately above. In these methods, a subject with the SNP genotype has an increased risk for manifesting schizophrenia over a subject with a different genotype.

The SNP identified as above is preferably associated with one of roh250, roh321, roh314, roh52, roh15, roh129, roh291, roh55, or roh173 as defined in Table 2.

The SNP identified as above can be within an open reading frame. Preferably, the open reading frame is in a gene selected from the group consisting of DYNC2H1, PIK3C3, CRHR1, IMP5, MAPT, STH, KIAA1267, LRRC37A, ARL17, LRRC37A2, NSF, WNT3, WNT9B, GOSR2, RPRML, CDC27, CHN1, ATF2, ATP5GS3, DUSP12, ATF6, OLFML2B, NOS1AP, SGCD, MRPL22, GPHN, C14orf54, MPP5, ATP6V1D, EIF2S1, PLEK2, GULP1, DIRC1, COL3A1, COL5A2, WDR75, SLC40A1, NS3TP1, ASNSD1, ANKAR, OSGEPL1, ORMDL1, PMS1, GDF8, and IMPAD1.

The identification of several genes within the schizophrenia-associated ROHs (Table 2) raises the possibility that a compound that affects the products of these genes affect schizophrenia. The invention is thus further directed to methods of screening for a compound that may affect schizophrenia. The methods comprise determining whether the compound affects expression or activity of a gene selected from the group consisting of DYNC2H1, CRHR1, IMP5, MAPT, STH, KIAA1267, LRRC37A, ARL17, LRRC37A2, WNT3, WNT9B, GOSR2, RPRML, CDC27, CHN1, ATP5GS3, DUSP12, ATF6, OLFML2B, SGCD, MRPL22, GPHN, C14orf54, MPP5, ATP6V1D, EIF2S1, PLEK2, GULP1, DIRC1, COL3A1, COL5A2, WDR75, SLC40A1, NS3TP1, ASNSD1, ANKAR, OSGEPL1, ORMDL1, PMS1, GDF8, IMPAD1, SNTG1 and SORCS1, Here, a compound that affects expression of the gene or activity of the gene product may affect schizophrenia. Preferred genes for these methods are MAPT, GPHN, SNTG1 and SORCS1.

In some aspects of these methods, the compound is contacted with a product of the gene then the activity of the gene product is measured. Alternatively, the compound is contacted with the product of the gene in vitro. In other aspects, the compound is contacted with a cell that expresses the product of the gene such that the compound contacts the product of the gene. Alternatively, the compound is contacted with a cell that is capable of expressing the gene, and expression of the gene is measured and compared to expression of the gene in a cell that is not contacted with the compound. In other aspects, the compound is administered to a mammal and activity of a product of the gene is measured and compared to activity of the product of the gene in a mammal that is not administered the compound. In further aspects, the compound is administered to a mammal and expression of the gene is measured and compared to expression of the gene in a mammal that is not administered the compound.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Example 1

### Runs of Homozygosity Reveal Highly Penetrant Recessive Loci in Schizophrenia

### Example Summary

Evolutionarily significant selective sweeps may result in long stretches of homozygous polymorphisms in individuals from outbred populations. Whole genome homozygosity association (WGHA) methodology was developed to exploit this phenomenon. This methodology was validated by identifying genetic risk loci for schizophrenia (SCZ). Applying WGHA to 178 SCZ cases and 144 healthy controls genotyped at 500,000 markers, it was found that runs of homozygosity (ROHs), ranging in size from 200 kb to 15 MB, were common in unrelated Caucasians. ROHs were significantly more common in SCZ, and a set of nine ROHs significantly differentiated cases from controls. Each of these 9 “risk ROHs” included genes relevant to post-synaptic structure and/or neuronal survival, and four contained or neighbored genes previously associated with SCZ (NOS1AP, ATF2, NSF, and PIK3C3). Results suggest that recessive effects of relatively high penetrance at CNS-relevant loci may explain a proportion of the genetic liability for SCZ.

### Introduction

Structural properties of whole genome association (WGA) datasets, including patterns of linkage disequilibrium (LD), have not yet been exploited in WGA analyses. Consequently, a novel analytic approach was developed, termed whole genome homozygosity association (WGHA). WGHA first identifies patterned clusters of SNPs demonstrating excess homozygosity and then employs both genomewide and regionally-specific statistical tests for association to disease. In the present study, WGHA was utilized in a case-control dataset of patients with schizophrenia (SCZ, MIM #181500) and healthy volunteers, genotyped at ˜500,000 SNPs, to detect novel susceptibility loci for SCZ.

WGHA (described in detail below) presents an opportunity for rapidly identifying susceptibility loci broadly across the genome, yet with resolution sufficient to implicate a circumscribed set of candidate genes. WGHA is designed to be sensitive for detecting loci under selective pressure, and recent data suggests that signatures of evolutionary selection may be strongly observed in genes regulating neurodevelopment (Williamson et al., 2007; Evans et al., 2005). Thus, WGHA may be particularly effective for a disorder such as SCZ, which is thought to have a primary pathophysiological basis in abnormal neurodevelopmental processes (Kamiya et al., 2005).

Regions of extended homozygosity across large numbers of consecutive SNPs form the basis of WGHA analysis. In general, extent of homozygosity is a function of LD within a chromosomal region, which in turn is a function of recombination rates and population history (McVean et al., 2004; Reich et al., 2002; Coop and Przeworski, 2007). Size and structure of LD blocks vary widely across the genome and across populations (Hinds et al., 2005), and regions of extensive long-range LD may be indicative of selective sweeps of functional significance (Kim and Nielson, 2004). For example, variants of the extended haplotype homozygosity test (Sabeti et al., 2002) have been used to examine identity-by-descent across unrelated chromosomes in HapMap (International HapMap Consortium, 2005) and other population samples, identifying known loci under selection (e.g., LCT in Europeans) (Voight et al., 2006; Wang et al., 2006). A logical consequence of such identity across unrelated chromosomes is that long stretches of homozygosity may be observed in healthy individuals from outbred populations lacking any known consanguineous parentage (Gibson et al., 2006; Simon-Sanchez et al., 2007). However, the relative commonality of this phenomenon has not been systematically documented in large datasets at high resolution. Moreover, while homozygosity mapping has successfully identified disease loci in pedigrees marked by Mendelian illness (Miyazawa et al., 2007), the ability of such a method to detect susceptibility loci in common disease has not been examined in a case-control study. Data is presented here addressing both normal patterns of homozygosity and use of these patterns in WGHA mapping of SCZ.

### Subjects and Methods

Participants. As described previously (Lencz et al., 2007), patients with SCZ spectrum disorders (total n=178, including 158 patients with schizophrenia, 13 patients with schizoaffective disorder, and 7 with schizophreniform disorder) were recruited from the inpatient and outpatient clinical services of The Zucker Hillside Hospital, a division of the North Shore-Long Island Jewish Health System. After providing written informed consent, the Structured Clinical Interview for DSM-IV Axis I disorders (SCID, version 2.0) was administered by trained raters. Information obtained from the SCID was supplemented by a review of medical records and interviews with family informants when possible; all diagnostic information was compiled into a narrative case summary and presented to a consensus diagnostic committee, consisting of a minimum of three senior faculty.

Healthy controls (n=144) were recruited by use of local newspaper advertisements, flyers, and community Internet resources and underwent initial telephone screening to assess eligibility criteria. After providing written informed consent, the nonpatient SCID (SCID-NP) was administered to subjects who met eligibility criteria, to rule out the presence of an Axis I psychiatric disorder; a urine toxicology screen for drug use and an assessment of the subject's family history of psychiatric disorders were also performed. Exclusion criteria included (current or past) Axis I psychiatric disorder, psychotropic drug treatment, substance abuse, a first-degree family member with an Axis I psychiatric disorder, or the inability to provide written informed consent. Patients (65 female/113 male) and controls (63F/81 M) did not significantly differ in sex distribution (P>0.05).

All subjects self-identified as Caucasian, non-Hispanic. As described previously (Lencz et al., 2007), population structure was tested by examination of 210 ancestry informative markers (AIMs). AIMs included all SNPs on the array that passed initial quality control procedures and demonstrated a frequency difference of ≧0.5 in comparisons between Caucasian individuals and Asians or African-Americans in data made publicly available by Shriver and colleagues (Shriver et al., 2003) (http://146.186.95.23/biolab/voyage/psa.html). Two tests of structure were performed, both of which indicated no significant stratification. First, analysis with the STRUCTURE program (Pritchard et al., 2000) confirmed that all subjects were drawn from a single population; second, comparison of cases and controls on allelic frequency across the 210 AIMs revealed no differences beyond those expected by chance.

Genotyping. Genomic DNA extracted from whole blood was hybridized to two oligonucleotide microarrays (Kennedy et al., 2003) containing ˜262,000 and ˜238,000 SNPs (mean spacing=5.8 kb; mean heterozygosity=27%) as per manufacturer's specifications (Affymetrix, Santa Clara, Calif.; S3). Genotype calls were obtained using the Bayesian Robust Linear Model with Mahalanobis distance classifier (BRLMM) algorithm thresholded at 0.5 applied to batches of 100 samples. Quality control procedures followed several steps (Lencz et al., 2007). First, samples that obtained mean call rates <90% across both chips (or <85% for a single chip) were rejected. Mean call rate of remaining samples (total n=322) was 97%. Twenty-two of these cases were successfully repeated, and concordance of the two calls (reliability) for each SNP was evaluated. SNPs with >1 discrepancy were excluded from further analyses. Concordance across the remaining 454,699 SNPs exceeded 99.4%. For WGHA, individual SNPs with low call rates even in valid cases were included, as were SNPs not in Hardy-Weinberg equilibrium in the control sample, because SNPs with these properties may be indicative of structural genomic variation of interest (McCarroll et al., 2006). However, 9936 SNPs in the sex-linked (i.e., non-pseudoautosomal) portion of the X chromosome were deleted, yielding 444,763 SNPs available for WGHA analysis. All statistical analyses described above were conducted using HelixTree software (Golden Helix, Inc., Bozeman, Mont.).

WGHA: Definitions and Statistical Analysis. WGHA analysis entails several within-subject and across-subject analytic steps, each performed with customized python scripting in the HelixTree environment, as follows. First, SNP data from each chromosome of each subject were interrogated for runs of homozygosity (ROHs), which are long series of consecutive SNPs that are homozygous (uncalled SNPs are permitted within a run, as these may indicate genomic phenomena of interest). A conservative threshold of 100 consecutive SNPs was selected to minimize false positive identification of ROHs occurring by chance (at the admitted risk of false negatives). Since mean heterozygosity across all SNPs was observed to be 27%, any given SNP has, on average, a 0.73 chance of being called homozygous. Given 444,763 reliable SNPs and 322 subjects, a minimum run length of 70 would be required to produce <5% family-wise error rate (i.e., randomly generated ROHs) across all subjects (0.73⁷⁰*444,763*322=0.04), assuming complete independence of all SNPs. Due to linkage disequilibrium, SNP calls are not fully independent, thereby inflating the likelihood of chance occurrence of biologically meaningless ROHs. Genomewide identification of tag SNPs within windows of 70 markers using the Carlson method (Carlson et al., 2004) as implemented in HelixTree revealed 314,869 separable tag groups, representing a 29.3% reduction of information compared to the total number of original SNPs. Thus, run size of 100 SNPs was selected to approximate the degrees of freedom of 70 independent SNP calls.

Each subject's SNP data were then converted to binary calls (0 or 1) at each position indicating whether that SNP is a member of an ROH for that individual. Next, at each position, data from all subjects was examined to determine whether a minimum number of individuals share an ROH call at a given position. Since the purpose of this investigation was the identification of statistical differences between biologically meaningful ROHs in a case-control design, SNPs with <10 ROH calls across the entire sample were eliminated, resulting in 65,422 SNPs with 10 or more ROH calls, an 85% reduction from the original pool of SNPs. Taking this strategy a step further, ‘common’ ROHs were identified which contained a minimum of 100 consecutive ROH calls across 10 or more subjects. A total of 339 such ROHs were identified across the genome, ranging in size from 100 to 852 SNPs in length (mean=161, SD=82, median=133, see Supplementary Table 1). A subject whose individual ROH calls overlapped with a common ROH was called ‘present’ for that common ROH. Thus, each subject could have a total (sum) score for presence of common ROHs ranging from 0 to 339.

Based on these definitions, the statistical plan followed several steps for the identification of differences between cases and controls. First, this total score for common ROHs was compared between cases and controls using Student's t-test; this constituted a single genomewide test for difference in ROH frequency, with a set to 0.05. Next, as a planned post-hoc examination of any significant genomewide difference, case-control comparisons of frequency of presence for each common ROH were examined using χ² tests (or Fisher's exact test when expected values <10 were found for any cell); although a would be protected by the preceding genomewide comparison, the threshold for significance for this analysis was set to p<0.01 to further reduce the risk of false positives. Third, the cumulative effect of these risk-imparting ROHs (i.e., the dose-dependence of the presence of “risk ROHs”) was tested with logistic regression. Because the predictor variables for these logistic regression analyses were the ROHs already identified as significantly differentiating cases and controls, the raw p-values for these regressions should be considered as strongly anti-conservative. Therefore, empirical p-values were calculated using 100,000 permutations of the full ROH dataset for each regression analysis.

Finally, as an exploratory analysis to potentially identify smaller regions of difference between cases and controls, χ² tests were performed on the 54,600 binarized SNP calls within common ROHs. Analogous to the dual-thresholding procedures commonly used in voxelwise brain imaging studies (Poline et al., 1997), statistical significance for these exploratory analyses was defined as 50 or more consecutive SNPs significantly differing between cases and controls at the p<0.01 level.

A summary version of the WGHA algorithm, as described above, is presented in pseudo-code form below. Assuming each subject is represented by a spreadsheet row and each QC-validated SNP on the microarray is represented by a spreadsheet column:
1) For each individual, scan across raw SNP data for runs of consecutive homozygous (or missing) calls >100 SNPs in length.
2) For each individual, recode each SNP call to a ‘0’ or ‘1’ indicating whether it is a member of an ROH for that individual.
3) Across subjects, scan down columns and delete all columns that contain fewer than ten 1's.
4) Construct a list of common ROHs by identifying, across all subjects, runs of ≧100 SNPs in length in which 10 or more subjects have consecutive 1's.
5) For each subject, mark each common ROH as ‘present’ if that subject contains any 1's within the boundaries of that ROH.
6) Conduct primary case-control analyses on scores derived from step 5 above. Genomewide analysis is conducted on the sum score across all ROHs. Given a significant genomewide case-control difference, individual ROHs can be examined for frequency differences to identify the source of this overall difference.
7) Conduct exploratory case-control analyses on binarized SNP scores derived from step 2 above. Significant case-control differences can be identified utilizing a two-step threshold (analogous to “height” and “extent” in voxelwise brain imaging studies): first, identify all SNPs at which case-control frequency differences are significant (p<0.01). Then, identify any runs of significant difference extending 50 or more SNPs in length. In the present study, this exploratory analysis resulted in the subregions listed in Supplementary Table 2.

### Results

As described above, the critical step of WGHA analysis is the identification of “common” runs of homozygosity” (ROHs) defined as those ROHs in which 10 or more subjects share ≧100 identical homozygous calls. Each common ROH was then scored “present” or “absent” for each subject. A total of 339 common ROHs were thus identified (Supplementary Table 1), encompassing approximately 12-13% of the genome as measured both by number of included SNPs and total chromosomal length. The six longest ROHs, ranging from 6 MB to 15.6 MB, encompass the centromeres of chromosomes 3, 5, 8, 11, 16, and 19. In part, this is a function of long regions with no SNPs ascertained; nevertheless, in each case, these centromeric gene deserts are flanked by homozygous regions containing hundreds of SNPs, possibly reflecting meiotic drive (Williamson et al., 2007). The greatest number of consecutive SNPs (852) is found in roh172, spanning the centromere of Chromosome 8; this region, which contains the gene encoding syntrophin gamma 1 (SNTG1), has been previously highlighted in several genomewide studies of selective sweeps (Williamson et al., 2007; International HapMap Consortium, 2005; Voight et al., 2006; Wang et al., 2006), thereby providing a positive control for our method.

There are 9 ROHs that were very common (>25% frequency) in healthy controls. As displayed in Table 1, publicly available data indicates that these regions are not marked by excessive copy number variation or segmental duplication. Moreover, these ROHs do not appear to have abnormally low recombination rates; the Phase II HapMap shows an average of about 5 recombination hotspots/MB across these 9 regions (International HapMap Consortium, 2005). On the other hand, examination of Haplotter data (Voight et al., 2006) (http://hg-wen.uchicago.edu/selection/haplotter.htm) indicates high scores for each of these regions on one or more measures of positive selection in Caucasian samples (iHS, Tajima's D, and/or Fst). Several gene categories previously identified in studies of selective pressure (Williamson et al., 2007; International HapMap Consortium, 2005; Voight et al., 2006; Wang et al., 2006) are evident in these regions, including genes involved in the immune system (on chromosomes 6p, 12q and 5q), olfactory receptors (6p and 11p), members of the dystrophin protein complex (SNTG1 and DGKZ), and many other CNS-expressed genes (e.g., GPI-IN, UNC5D, ATXN2).

TABLE 1
List of 9 ROHs with frequency ≧25% in the healthy cohort (n = 144). Chromosomal coordinates listed from NCBI Build 35. Columns I,
J, and K represent maximal values for alternate metrics of positive selection, derived from Haplotter (ref. 15, http://hg-wen.uchicago.edu/
selection/haplotter.htm). Number of deletions and duplications contained within each region derived from ref. 30
(http://projects.tcag.ca/variation). Percentage of each ROH containing segmental duplication and number of recombination
hotspots estimated from HapMap version 21a (ref. 14, http://hapmap.org).

| | | | | Length | # | N | % | iHS | TajimaD | Fst | # Deletions/ | % | # |
|---|---|---|---|---|---|---|---|---|---|---|---|---|---|
| ROH | Chr | Start (B35) | End (B35) | (bp) | SNPs | Control | Control | max | max | max | # Duplications | Seg_Dup | HotSpots |
| roh172 | 8 | 42588087 | 53273605 | 10685518 | 852 | 77 | 53.5 | 3.90 | 2.00 | 0.90 | 0/6 | 0.00 | 32.00 |
| roh134 | 6 | 26216147 | 29800284 | 3584137 | 489 | 52 | 36.1 | 2.40 | 1.50 | 0.52 | 0/3 | 0.10 | 13.00 |
| roh89 | 4 | 32428277 | 34888919 | 2460642 | 252 | 52 | 36.1 | 3.50 | 2.40 | 0.62 | 9/2 | 0.05 | 17.00 |
| roh241 | 11 | 46212732 | 49874378 | 3661646 | 323 | 48 | 33.3 | 2.30 | 1.75 | 0.70 | 11/1 | 0.35 | 3.00 |
| roh291 | 14 | 65475183 | 67065410 | 1590227 | 197 | 47 | 32.6 | 2.50 | 2.75 | 0.99 | 0/0 | 0.00 | 9.00 |
| roh171 | 8 | 33590815 | 36749387 | 3158572 | 443 | 41 | 28.5 | 0.60 | 2.80 | 0.42 | 0/0 | 0.00 | 19.00 |
| roh238 | 11 | 37492528 | 40090659 | 2598131 | 386 | 39 | 27.1 | 1.10 | 5.10 | 0.45 | 0/0 | 0.00 | 25.00 |
| roh275 | 12 | 109752647 | 111733445 | 1980798 | 205 | 37 | 25.7 | 1.10 | 1.80 | 0.22 | 0/0 | 0.00 | 8.00 |
| roh125 | 5 | 129481382 | 132022568 | 2541186 | 286 | 36 | 25.0 | 1.20 | 1.25 | 0.65 | 1/1 | 0.00 | 12.00 |

The total number of common ROHs marked “present” was summed for each subject to permit genomewide comparison across diagnostic groups, prior to group comparisons of frequency of individual ROHs. Out of a total possible sum score of 339, patients with schizophrenia demonstrated a significantly greater number of common ROHs scored ‘present’ (mean=31.7, SD=12.3) relative to healthy volunteers (mean=28.0, SD=12.8; t₃₂₀=2.62, P=0.009). Nine individual ROHs significantly <0.01) differed in frequency between cases and controls (Table 2); each was more common in SCZ cases.

TABLE 2
List of 9 risk ROHs significantly over-represented in SCZ cases (p < .01). Genes previously associated with SCZ listed in bold.

| | | | | | | N | % | N | % | | | |
|---|---|---|---|---|---|---|---|---|---|---|---|---|
| ROH | Chr | Start (B35) | End (B35) | Length | # SNPs | Cases | Cases | Control | Control | X² | P | Genes |
| roh250 | 11 | 102488778 | 102947117 | 458339 | 103 | 14 | 7.9 | 0 | 0.0 | Fisher | 0.0004 | DYNC2H1 |
| roh321 | 18 | 37022928 | 37619977 | 597049 | 119 | 15 | 8.4 | 1 | 0.7 | Fisher | 0.0012 | (PIK3C3) |
| roh314 | 17 | 41169023 | 42622984 | 1453961 | 211 | 40 | 22.5 | 14 | 9.7 | 9.271 | 0.0023 | CRHR1; IMP5; MAPT; STH; |
| | | | | | | | | | | | | KIAA1267; LRRC37A; |
| | | | | | | | | | | | | ARL17; LRRC37A2; NSF; |
| | | | | | | | | | | | | WNT3; WNT9B; GOSR2; |
| | | | | | | | | | | | | RPRML; CDC27 |
| roh52 | 2 | 175671012 | 176445047 | 774035 | 115 | 17 | 9.6 | 2 | 1.4 | Fisher | 0.0032 | CHN1; ATF2; ATP5GS3 |
| roh15 | 1 | 158440777 | 159015569 | 574792 | 173 | 20 | 11.2 | 4 | 2.8 | 8.256 | 0.0041 | DUSP12; ATF6; OLFML2B; |
| | | | | | | | | | | | | NOS1AP |
| roh129 | 5 | 154592379 | 155033077 | 440698 | 116 | 13 | 7.3 | 1 | 0.7 | Fisher | 0.0042 | (SGCD, MRPL22) |
| roh291 | 14 | 65475183 | 67065410 | 1590227 | 197 | 86 | 48.3 | 47 | 32.6 | 8.068 | 0.0045 | GPHN; C14orf54; MPP5; |
| | | | | | | | | | | | | ATP6V1D; EIF2S1; PLEK2 |
| roh55 | 2 | 188489676 | 190772106 | 2282430 | 274 | 31 | 17.4 | 10 | 6.9 | 7.855 | 0.0051 | GULP1; DIRC1; COL3A1; |
| | | | | | | | | | | | | COL5A2; WDR75; |
| | | | | | | | | | | | | SLC40A1; NS3TP1; |
| | | | | | | | | | | | | ASNSD1; ANKAR; |
| | | | | | | | | | | | | OSGEPL1; ORMDL1; PMS1; |
| | | | | | | | | | | | | GDF8 |
| roh173 | 8 | 57989122 | 58616467 | 627345 | 120 | 20 | 11.2 | 5 | 3.5 | 6.7 | 0.0096 | IMPAD1 |

Several features of these 9 “risk ROHs” are notable. First, presence of the risk ROHs is not common in healthy subjects, and presence of several is exceedingly rare in healthy subjects (Table 3). Greater than half (54.9%) of healthy controls, but only 19.1% of SCZ subjects, did not have any risk ROHs present in their WGHA data (χ²=44.7, df=1, P=2.3*10⁻¹¹; permuted P=0.0022; Odds Ratio=5.15, 95% CI=3.13-8.46). Moreover, as the number of risk ROHs increases, risk of illness increases dramatically. Using logistic regression, total number of risk ROHs significantly predicted group status (χ²=62.6, df=1, P=2.51*10⁻¹⁵; permuted P=0.00095; with each additional risk ROH imparting an odds ratio of 2.83 (95% CI=2.10-3.81, see also Table 3).

TABLE 3
Odds of SCZ as a function of number of risk ROHs present in a given
individual. Odds ratios computed using sum = 0 as reference category.
For purposes of calculation, individuals with ≧3 risk ROHs were
grouped together.

| #Risk | N | | | | | |
|---|---|---|---|---|---|---|
| ROHs (Sum) | Cases | % Cases | N Control | % Control | OR* | 95% CI |
| 0 | 34 | 19.1 | 79 | 54.9% | | |
| 1 | 70 | 39.3 | 49 | 34.0% | 3.3 | 1.9-5.7 |
| 2 | 43 | 24.2 | 13 | 9.0% | 5.4 | 3.7-16.1 |
| 3 | 25 | 14.0 | 3 | 2.1% | 24** | 6.9-83.9 |
| 4 | 5 | 2.8 | 0 | 0.0% | | |
| 5 | 1 | 0.6 | 0 | 0.0% | | |

*OR compared to Sum = 0.
**Sum ≧3 compared to Sum = 0.

Six of the nine risk ROHs listed in Table 2 are extremely rare in healthy controls. One ROH (roh250), containing the gene encoding the dynein cytoplasmic 2, heavy chain 1 protein (DYNC2H1 on chromosome 11 q), was exclusively observed in SCZ; in other words, this genetic variant demonstrated 100% penetrance for illness. On the other hand, one very common ROH in healthy subjects (roh291) also conferred risk for SCZ (χ²=8.1, df=1, P=0.0045). This ROH is centered on the very large (˜675 kb) gene GPHN, which codes for gephyrin, a protein scaffold that serves to anchor GABA receptors in the postsynaptic membrane.

As with GPHN, the genes implicated in all but one of these regions (roh55) are amenable to neurodevelopmental interpretations consistent with known or hypothesized SCZ pathophysiological mechanisms (Kamiya et al., 2005). Specifically, roh15 on chromosome 1q contains NOS1AP (formerly CAPON), which has been related to schizophrenia in both genetic linkage and association studies, as well as in post-mortem gene expression studies (Brzustowicz et al., 2004; Zeng et al., 2005; Xu et al., 2005). This protein competes with PSD95 for binding to neuronal nitric oxide synthase (nNOS), thereby disrupting neuronal NMDA receptor transmission at the post-synaptic density. Similarly, roh52 contains ATF2, a downstream target of the mitogen-activated protein kinase/extracellular signal-regulated kinase signaling pathway triggered by nNOS; protein levels of activating transcription factor 2 have been reported to be elevated in postmortem SCZ brain tissue (Kyosseva et al., 2000). Further, roh314 contains NSF (encoding a critical presynaptic protein, N-ethylmaleimide sensitive fusion), which regulates dissociation of the SNARE complex and binds to the GluR2 subunit of AMPA glutamate receptors. Abnormalities in this gene have been also linked with schizophrenia in both gene expression and genetic association studies (Mimics et al., 2000; Allen et al., 2007). In addition to NSF, roh314 (at chromosome 17q21) contains MAPT (microtubule-associated protein tau). MAPT has been previously reported to contain a common inversion under selective pressure, resulting in a distinctive haplotypic genealogy that has been associated with multiple neurological disorders, including Alzheimer's disease, fronto-temporal dementia, and progressive supranuclear palsy (Hardy et al., 2006).

Two ROHs which were significantly over-represented in patients with SCZ contained no known genes (roh321 on chromosome 18q and roh129 on 5q). While both regions include one or more ESTs and may harbor as-yet unknown regulatory elements, it is also possible that extensive allelic hitchhiking may result in effects on genes immediately neighboring these regions (McVean et al., 2004). Consequently, the first gene located within 500 kb in either direction of these ROHs is listed in parentheses in Table 2. PIK3C3 (adjacent to roh129) encodes phosphoinositide-3-kinase, class 3, which is highly expressed throughout the brain. A promoter region variant in this gene has been associated with SCZ in three studies to date (Allen et al., 2007). Moreover, the PI3K/AKT signaling cascade modulates activation of ErbB4 receptors in oligodendrocytes, which are activated by neuregulin, widely considered a SCZ risk gene (Allen et al., 2007; Law et al., 2007).

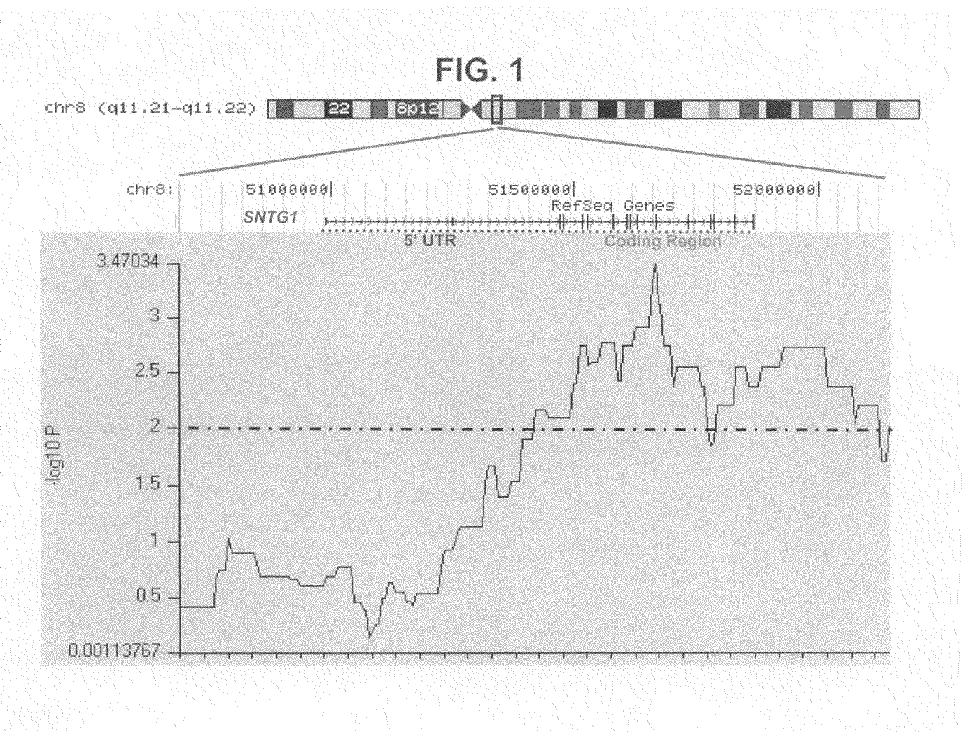

Finally, exploratory analyses examining binarized individual SNP data revealed subregions of two additional ROHs which were significantly over-represented in SCZ cases relative to controls (Supplementary Table 2). Segments of the very large ROH on chromosome 8 (roh172), demonstrated a strong differentiation between cases and controls (maximal χ²=12.9, df=1, P=3.28*10⁴) occurring directly in the coding region of SNTG1 (FIG. 1). SNTG1 is expressed exclusively in neurons, including hippocampal pyramidal cells, cerebellar Purkinje cells, and multiple cortical regions, where it binds to dystrophin, the dystrobrevins, and diacylglycerol kinase, zeta (DGKZ) in the post-synaptic density. SORCS1, which is widespread throughout the brain and has been recently characterized as a gamma-secretase substrate, was also identified as a significant subregion of an ROH on chromosome 10q.

### Discussion

Taken together, these data suggest the utility of WGHA in identifying disease-relevant genomic regions of interest, and support several hypotheses concerning the genetic architecture of SCZ. Utilizing dense, whole-genome microarray SNP data, we observed that runs of homozygosity ranging in size from 200 kb to more than 15 MB were common even in healthy individuals from an outbred population (U.S. Caucasians residing in New York City/Long Island). These homozygous regions are both too common and too small to suggest recent consanguineity. Rather, convergence with prior reports suggests that ROHs mark regions under selective pressure. The most common ROHs in the present study (Table 1) have generally been implicated in prior studies using varying coalescent models and statistical assumptions (Williamson et al., 2007; International HapMap Consortium, 2005; Voight et al., 2006; Wang et all, 2006). At the same time, genes recognized by other methods as under strong selective pressure in Caucasians, such as SNTG1 (included in roh172), ALDH2 (roh275), LCT (roh45), and SLC24A5 (roh296), are successfully captured amongst the common ROHs listed in Supplementary Table 1.

Because the SNP selection of the current generation of whole-genome microarrays is still limited and does not permit uniform coverage across the genome, the likelihood of SNP ascertainment bias limits formal statistical testing of the evidence for selection (Clark et al., 2005). However, relative frequency of these ROHs in an unselected, healthy Caucasian population provides a metric that is significantly correlated with other measures of selection (Voight et al., 2006). Across regions, ROH frequency in controls was significantly correlated with maximal iHS (r=0.33, P=3.4*10⁻¹⁰) and Tajima's D (r=0.30, P=2.8*10⁻⁸); these correlations are comparable to the intercorrelation of maximal iHS and D for the same regions (r=0.30, P=1.3*10⁻⁸). Moreover, ROH frequency is a readily available measure for statistical comparisons in a case-control design. Thus, current and future generations of commercially available genotyping microarrays can provide evolutionarily-meaningful data at the genomic level.

We also observed that ROHs were over-represented in SCZ cases at a genomewide level, and that presence of nine specific ROHs was associated with illness susceptibility both individually and cumulatively. Intriguingly, genes found in these regions tended to converge upon a limited number of CNS-relevant pathways. Four of these regions implicated genes related to post-synaptic (largely glutamatergic) receptor complexes previously implicated in SCZ pathophysiology. These genes include NOS1AP and NSF, each of which has been previously associated with schizophrenia, as well as GPHN and SGCD, which have not been previously examined in SCZ association studies. A fifth region spanning the coding region of SNTG1 was associated with SCZ in exploratory analyses; syntrophin abnormalities in SCZ are consistent with the accumulating evidence associating DTNBP1 haplotypic variation with SCZ susceptibility (Allen et al., 2007; Funke et al., 2004).

Five risk ROHs (including one identified in the exploratory analysis) contain or neighbor genes related to neuronal proliferation and survival, either via the phosphatidylinositol signaling pathway (IMPAD1 and PIK3C3), activating transcription factors (ATF2 and ATF6), or through binding with growth factors (SORCS1). Additionally, it is notable that the risk ROH with the strongest association to schizophrenia contained only one gene, encoding a dynein subunit. Although DYNC2H1 is not as well characterized as other cytoplasmic dynein subunits (which bind with the well-studied schizophrenia risk gene DISCI [Kamiya et al., 2005; Allen et al., 2007; Hodgkinson et al., 2004]), the implication of microtubule dysgenesis is consistent with current pathophysiological hypotheses in SCZ⁷, and converges with the implication of MAPT in an additional risk ROH.

It should be noted that results for the MAPT region may be influenced by the frequent presence of copy number variation at chromosome 17q21 (Redon et al., 2006); however, it is unlikely that results of the present study are primarily reflective of copy number variation, for four reasons. First, HapMap data suggests that duplications in this region are far more common than deletions (Redon et al., 2006), whereas deletions are more likely to create a spurious pattern of homozygous calls (McCarroll et al., 2006). Second, deletions in this region have been associated with mental retardation (Sharp et al., 2006), which is not observed in our study. Third, chromosomal locations containing highly common ROHs (Table 1) are not generally marked by frequent copy number variation in publicly available databases (Redon et al., 2006). Fourth, inspection of raw intensity plots from microarrays analyzed for the present study are not consistent with frequent, large regions of copy number variation in the neighborhood of common ROHs (data not shown). Further research is needed to carefully examine the role of copy number variation in SCZ.

Finally, if ROHs provide an index of genomic regions undergoing positive selection, it is perhaps counterintuitive that ROHs would be more commonly observed in patients with schizophrenia. However, results are consistent with a model of rare, deleterious recessive effects associated with an allele or haplotype with positive co-dominant properties (Voight et al., 2006). These balancing effects may either be the result of the same allele, as in HBB and malaria, or from distal alleles that have hitchhiked near a region undergoing selection. It has been suggested that an example of the latter is hereditary hemochromatosis, a relatively common recessive disorder involving a mutation in HFE. While some evidence suggests that the FIFE mutation itself is subject to balancing selection, a recent genomewide scan for evolutionary signal indicated that positive selection was more likely to be acting on the adjacent large histone cluster at chromosome 6p22 (Williamson et al., 2007); this histone cluster (but not HFE) was also the site of a relatively common ROH (roh134) in the present study. While WGHA currently lacks the spatial resolution to identify the causative allele(s), regions reported in the present study provide fairly narrow windows containing highly plausible candidates for further investigation. They also suggest that recessive effects of relatively high penetrance at multiple loci may explain a proportion of the genetic liability for SCZ.

SUPPLEMENTARY TABLE 1
List of all 339 common ROHs. Start and stop positions are listed as a function of chromosomal position (both Build 35 and Build
34 coordinates are presented), SNP Affymetrix ID's, and SNP rs numbers.

| ROH | Chr | Start_Cyto | Stop_Cyto | Start_pos | End_pos | Length | #SNPs | Start_Affy | End_Affy | Start_rs |
|---|---|---|---|---|---|---|---|---|---|---|
| roh2 | 1 | p35.2 | p35.2 | 31180097 | 31783886 | 603789 | 107 | A-2296736 | A-2000348 | rs7537241 |
| roh3 | 1 | p34.3 | p34.3 | 34972868 | 36573853 | 1600985 | 118 | A-4256194 | A-4219724 | rs6655940 |
| roh4 | 1 | p34.1 | p34.1 | 45113131 | 46347229 | 1234098 | 115 | A-1913736 | A-2030685 | rs11556200 |
| roh5 | 1 | p33 | p32.3 | 48801467 | 50466576 | 1665109 | 182 | A-4241953 | A-2261210 | rs11807595 |
| roh6 | 1 | p32.3 | p32.2 | 55603820 | 56532672 | 928852 | 245 | A-2041390 | A-2026309 | rs11206580 |
| roh7 | 1 | p31.1 | p31.1 | 72245948 | 73882032 | 1636084 | 209 | A-1898528 | A-2277303 | rs2630380 |
| roh8 | 1 | p31.1 | p31.1 | 75613158 | 76201800 | 588642 | 117 | A-2295414 | A-1898797 | rs17096877 |
| roh9 | 1 | p31.1 | p31.1 | 80022979 | 80639208 | 616229 | 115 | A-1849732 | A-2267047 | rs17103827 |
| roh10 | 1 | p22.2 | p22.2 | 88567123 | 89184874 | 617751 | 103 | A-1938599 | A-4285123 | rs443499 |
| roh11 | 1 | p21.3 | p21.3 | 96307200 | 97160204 | 853004 | 131 | A-2071697 | A-1854765 | rs1222069 |
| roh12 | 1 | p21.2 | p21.2 | 99997212 | 100910847 | 913635 | 123 | A-2283986 | A-1849514 | rs11166349 |
| roh13 | 1 | p21.1 | p21.1 | 102463733 | 103469102 | 1005369 | 133 | A-4244090 | A-1907564 | rs1578575 |
| roh14 | 1 | q21.3 | q21.3 | 149214350 | 150010691 | 796341 | 199 | A-1855975 | A-2160307 | rs6587671 |
| roh15 | 1 | q23.3 | q23.3 | 158440777 | 159015569 | 574792 | 173 | A-1901847 | A-1969201 | rs1503814 |
| roh16 | 1 | q23.3 | q23.3 | 160689111 | 161146399 | 457288 | 103 | A-2190181 | A-1823445 | rs16823642 |
| roh17 | 1 | q24.1 | q24.1 | 163223096 | 163719936 | 496840 | 106 | A-2199679 | A-2181943 | rs1021499 |
| roh18 | 1 | q24.2 | q24.2 | 166355252 | 166835037 | 479785 | 125 | A-2203047 | A-4263351 | rs7535059 |
| roh19 | 1 | q25.1 | q25.1 | 170121297 | 171823807 | 1702510 | 168 | A-2000482 | A-4251147 | rs4294450 |
| roh20 | 1 | q25.1 | q25.2 | 172525946 | 173746872 | 1220926 | 187 | A-1790335 | A-1826369 | rs476146 |
| roh21 | 1 | q25.3 | q25.3 | 179311585 | 180284983 | 973398 | 156 | A-2178976 | A-1859825 | rs609990 |
| roh22 | 1 | q25.3 | q31.1 | 182247118 | 182909895 | 662777 | 114 | A-1975823 | A-4299638 | rs1321996 |
| roh23 | 1 | q31.1 | q31.1 | 185525129 | 186689757 | 1164628 | 131 | A-2000558 | A-1908476 | rs7519600 |
| roh24 | 1 | q31.1 | q31.2 | 186802518 | 187568490 | 765972 | 116 | A-1977181 | A-1870345 | rs16832009 |
| roh25 | 1 | q31.3 | q31.3 | 190656365 | 191449984 | 793619 | 116 | A-1914869 | A-2064189 | rs12353935 |
| roh26 | 1 | q41 | q41 | 216196369 | 216833320 | 636951 | 113 | A-4226649 | A-2219189 | rs17006203 |
| roh27 | 1 | q42.12 | q42.12 | 220962951 | 221800318 | 837367 | 130 | A-2036007 | A-4265758 | rs10753470 |
| roh28 | 1 | q43 | q43 | 232999736 | 233305452 | 305716 | 106 | A-2076452 | A-2235968 | rs16833729 |
| roh29 | 2 | p24.2 | p24.2 | 17170201 | 17921554 | 751353 | 133 | A-1962289 | A-2024711 | rs7592574 |
| roh30 | 2 | p24.1 | p24.1 | 21376150 | 22452456 | 1076306 | 154 | A-1917234 | A-2074117 | rs312042 |
| roh31 | 2 | p22.1 | p22.1 | 38866070 | 39865134 | 999064 | 145 | A-2204016 | A-1962860 | rs11124646 |
| roh32 | 2 | p22.1 | p22.1 | 39979675 | 40403864 | 424189 | 130 | A-2136038 | A-1941421 | rs3953508 |
| roh33 | 2 | p22.1 | p21 | 41472377 | 41814193 | 341816 | 104 | A-1870597 | A-4270895 | rs6750424 |
| roh34 | 2 | p16.3 | p16.2 | 52794897 | 53269856 | 474959 | 123 | A-1903359 | A-4251321 | rs1454402 |
| roh35 | 2 | p16.1 | p16.1 | 57678029 | 58404092 | 726063 | 115 | A-2043789 | A-4236460 | rs4671309 |
| roh36 | 2 | p12 | p12 | 81577055 | 82119607 | 542552 | 108 | A-2161985 | A-1956032 | rs17020238 |
| roh37 | 2 | p12 | p11.2 | 82180277 | 84977903 | 2797626 | 414 | A-2234661 | A-2106559 | rs1521690 |
| roh38 | 2 | p11.2 | p11.2 | 86006859 | 86786032 | 779173 | 152 | A-4304347 | A-2006529 | rs4553826 |
| roh39 | 2 | q11.2 | q12.1 | 102286014 | 102739996 | 453982 | 140 | A-1964285 | A-4208263 | rs11686153 |
| roh40 | 2 | q14.1 | q14.1 | 114251872 | 114932937 | 681065 | 105 | A-1851194 | A-1826581 | rs17197583 |
| roh41 | 2 | q14.1 | q14.1 | 115472662 | 116531030 | 1058368 | 165 | A-1794278 | A-2039164 | rs1980006 |
| roh42 | 2 | q14.1 | q14.2 | 116951313 | 118734753 | 1783440 | 355 | A-4279966 | A-1888280 | rs12988073 |
| roh43 | 2 | q14.3 | q14.3 | 123521145 | 123995227 | 474082 | 106 | A-2182663 | A-2101140 | rs953374 |
| roh44 | 2 | q21.1 | q21.1 | 129683864 | 130251973 | 568109 | 135 | A-2302355 | A-2187128 | rs1441138 |
| roh45 | 2 | q21.3 | q22.1 | 135108498 | 137633548 | 2525050 | 359 | A-1964965 | A-2128393 | rs669746 |
| roh46 | 2 | q23.3 | q23.3 | 151695695 | 153239917 | 1544222 | 292 | A-2023366 | A-2217773 | rs7598311 |
| roh47 | 2 | q24.1 | q24.1 | 157714731 | 158629275 | 914544 | 103 | A-4303795 | A-4219570 | rs16841208 |
| roh48 | 2 | q24.1 | q24.1 | 158716270 | 159414982 | 698712 | 121 | A-2276336 | A-2155035 | rs6731028 |
| roh49 | 2 | q24.2 | q24.2 | 160890092 | 162616986 | 1726894 | 230 | A-1965588 | A-2079310 | rs7588696 |
| roh50 | 2 | q24.2 | q24.3 | 162741920 | 163656867 | 914947 | 118 | A-1965618 | A-4239001 | rs10490422 |
| roh51 | 2 | q24.3 | q24.3 | 167319757 | 168222033 | 902276 | 122 | A-1948272 | A-4208477 | rs10930239 |
| roh52 | 2 | q31.1 | q31.1 | 175671012 | 176445047 | 774035 | 115 | A-1884840 | A-2231272 | rs6433512 |
| roh53 | 2 | q31.1 | q31.2 | 177760878 | 178868836 | 1107958 | 232 | A-2090355 | A-2167861 | rs17683574 |
| roh54 | 2 | q32.1 | q32.1 | 185850874 | 188141083 | 2290209 | 277 | A-1966200 | A-1966232 | rs9288109 |
| roh55 | 2 | q32.1 | q32.2 | 188489676 | 190772106 | 2282430 | 274 | A-2281506 | A-1966267 | rs6434236 |
| roh56 | 2 | q32.3 | q33.1 | 193172893 | 197903456 | 4730563 | 581 | A-1966317 | A-1966408 | rs1471772 |
| roh57 | 2 | q33.1 | q33.1 | 199491617 | 200392642 | 901025 | 137 | A-2209572 | A-4198421 | rs13035389 |
| roh58 | 2 | q34 | q34 | 209978726 | 210836492 | 857766 | 110 | A-2134859 | A-2202879 | rs12614235 |
| roh59 | 2 | q35 | q35 | 218048529 | 218463682 | 415153 | 103 | A-2120160 | A-1966943 | rs4674159 |
| roh60 | 2 | q35 | q35 | 219054652 | 219977864 | 923212 | 109 | A-2265298 | A-2081914 | rs4674297 |
| roh61 | 2 | q36.1 | q36.2 | 224704712 | 225123617 | 418905 | 105 | A-4298175 | A-2199994 | rs4674839 |
| roh62 | 3 | p26.2 | p26.1 | 3744308 | 4081603 | 337295 | 110 | A-4252347 | A-1903535 | rs809958 |
| roh63 | 3 | p22.1 | p22.1 | 40029744 | 40992074 | 962330 | 126 | A-1874319 | A-4292507 | rs4234128 |
| roh64 | 3 | p22.1 | p22.1 | 41078249 | 42158780 | 1080531 | 180 | A-2306677 | A-2154578 | rs404477 |
| roh65 | 3 | p22.1 | p21.33 | 43173803 | 43775885 | 602082 | 116 | A-2106913 | A-1972537 | rs6442140 |
| roh66 | 3 | p21.31 | p21.31 | 45807056 | 46359173 | 552117 | 105 | A-1972582 | A-2162405 | rs1860263 |
| roh67 | 3 | p21.31 | p21.31 | 46524192 | 48366512 | 1842320 | 141 | A-4192504 | A-2105231 | rs7371865 |
| roh68 | 3 | p21.31 | p21.1 | 49434256 | 53139843 | 3705587 | 294 | A-1786118 | A-4253415 | rs1464567 |
| roh69 | 3 | p14.3 | p14.3 | 55992638 | 56508056 | 515418 | 101 | A-2105090 | A-1911674 | rs9878600 |
| roh70 | 3 | p14.1 | p14.1 | 67604470 | 68137649 | 533179 | 100 | A-1851658 | A-1941779 | rs9848013 |
| roh71 | 3 | p14.1 | p14.1 | 68414301 | 69128335 | 714034 | 152 | A-2094279 | A-2233334 | rs902719 |
| roh72 | 3 | p12.3 | p12.3 | 74628467 | 75535509 | 907042 | 102 | A-1838071 | A-1857574 | rs6549604 |
| roh73 | 3 | p12.3 | p12.2 | 79043731 | 81079810 | 2036079 | 224 | A-2050990 | A-1824316 | rs17016593 |
| roh74 | 3 | p12.2 | p12.2 | 81468557 | 82529436 | 1060879 | 129 | A-2080661 | A-2114119 | rs6796596 |
| roh75 | 3 | p12.1 | p12.1 | 85226226 | 86336795 | 1110569 | 129 | A-2257192 | A-2109268 | rs7623714 |
| roh76 | 3 | p12.1 | p11.1 | 86843760 | 87903799 | 1060039 | 133 | A-1870899 | A-1831150 | rs9850360 |
| roh77 | 3 | p11.1 | q11.2 | 89122747 | 98869488 | 9746741 | 470 | A-2102051 | A-4204556 | rs12495112 |
| roh78 | 3 | q12.1 | q12.1 | 100199144 | 101331030 | 1131886 | 197 | A-4237725 | A-2089071 | rs9852455 |
| roh79 | 3 | q13.13 | q13.13 | 111474429 | 112720470 | 1246041 | 202 | A-1821106 | A-1876313 | rs2712998 |
| roh80 | 3 | q13.31 | q13.31 | 117218506 | 117891571 | 673065 | 105 | A-2271977 | A-2256419 | rs16824392 |
| roh81 | 3 | q21.1 | q21.2 | 124609796 | 125505655 | 895859 | 153 | A-1974225 | A-1914214 | rs9840967 |
| roh82 | 3 | q25.32 | q25.32 | 159036150 | 159904511 | 868361 | 104 | A-2149135 | A-4205170 | rs1515473 |
| roh83 | 3 | q26.1 | q26.1 | 163807929 | 164922430 | 1114501 | 143 | A-2219850 | A-2060638 | rs7611213 |
| roh84 | 3 | q26.1 | q26.1 | 166843719 | 167611821 | 768102 | 110 | A-2135182 | A-2172913 | rs520325 |
| roh85 | 3 | q26.33 | q26.33 | 181062963 | 182033404 | 970441 | 118 | A-2044229 | A-2079712 | rs12638061 |
| roh86 | 4 | p16.2 | p16.2 | 5372730 | 5711417 | 338687 | 100 | A-2185882 | A-1907465 | rs11942948 |
| roh87 | 4 | p16.1 | p16.1 | 9503026 | 10205292 | 702266 | 274 | A-2076959 | A-4193779 | rs6822889 |
| roh88 | 4 | p15.33 | p15.33 | 14071587 | 14466747 | 395160 | 108 | A-1801230 | A-4287108 | rs735855 |
| roh89 | 4 | p15.1 | p15.1 | 32428277 | 34888919 | 2460642 | 252 | A-2063638 | A-1927293 | rs10003290 |
| roh90 | 4 | p14 | p14 | 36553147 | 36963199 | 410052 | 101 | A-2236945 | A-2075284 | rs11096832 |
| roh91 | 4 | p13 | p13 | 42965638 | 43803497 | 837859 | 113 | A-2076542 | A-4281964 | rs1436485 |
| roh92 | 4 | p11 | q12 | 49350542 | 53460295 | 4109753 | 126 | A-4217519 | A-4222283 | rs6668639 |
| roh93 | 4 | q13.1 | q13.1 | 60662524 | 61535046 | 872522 | 151 | A-4264240 | A-2287685 | rs349235 |
| roh94 | 4 | q13.1 | q13.1 | 65468989 | 66087039 | 618050 | 113 | A-1917314 | A-1977422 | rs17085296 |
| roh95 | 4 | q13.3 | q13.3 | 72284202 | 72963446 | 679244 | 100 | A-4210270 | A-2180391 | rs4694087 |
| roh96 | 4 | q13.3 | q13.3 | 74634723 | 75447147 | 812424 | 139 | A-2263880 | A-1837531 | rs3756066 |
| roh97 | 4 | q21.1 | q21.1 | 78459262 | 78829924 | 370662 | 174 | A-4264296 | A-4264308 | rs17002416 |
| roh98 | 4 | q21.21 | q21.21 | 79434452 | 80244222 | 809770 | 153 | A-4264312 | A-1954571 | rs10518193 |
| roh99 | 4 | q21.21 | q21.21 | 81571295 | 82481513 | 910218 | 113 | A-1977776 | A-4226672 | rs2290755 |
| roh100 | 4 | q26 | q26 | 118942583 | 120138941 | 1196358 | 141 | A-2056016 | A-2152154 | rs2635208 |
| roh101 | 4 | q26 | q27 | 120299115 | 121104478 | 805363 | 133 | A-1978673 | A-2142882 | rs10518312 |
| roh102 | 4 | q28.1 | q28.1 | 127867933 | 129109206 | 1241273 | 173 | A-2043112 | A-1978888 | rs313104 |
| roh103 | 4 | q28.2 | q28.2 | 130744909 | 131324225 | 579316 | 105 | A-1918836 | A-1978923 | ? |
| roh104 | 4 | q31.21 | q31.21 | 143056946 | 144889596 | 1832650 | 283 | A-1860835 | A-1877357 | rs9996010 |
| roh105 | 4 | q31.22 | q31.22 | 147801142 | 148686553 | 885411 | 157 | A-4294954 | A-1847909 | ? |
| roh106 | 4 | q31.23 | q31.3 | 151664818 | 152966894 | 1302076 | 131 | A-2168247 | A-4234973 | rs17504366 |
| roh107 | 4 | q31.3 | q31.3 | 154979551 | 155240449 | 260898 | 103 | A-2246036 | A-2281352 | rs11938228 |
| roh108 | 4 | q32.1 | q32.1 | 159144707 | 159986756 | 842049 | 126 | A-4285187 | A-1793439 | rs6813626 |
| roh109 | 4 | q33 | q33 | 171050376 | 171590580 | 540204 | 106 | A-1836416 | A-2122782 | rs17055122 |
| roh110 | 4 | q33 | q34.1 | 171639721 | 172981468 | 1341747 | 195 | A-2244734 | A-2101661 | rs11724276 |
| roh111 | 4 | q34.1 | q34.1 | 173568500 | 174320460 | 751960 | 104 | A-2262388 | A-4272722 | rs7699559 |
| roh112 | 5 | p15.2 | p15.2 | 12101652 | 13430951 | 1329299 | 135 | A-1867351 | A-2271092 | rs12517202 |
| roh113 | 5 | p14.2 | p14.2 | 23502023 | 24547699 | 1045676 | 139 | A-2055283 | A-1817466 | rs17313823 |
| roh114 | 5 | p13.1 | p13.1 | 39618799 | 40263343 | 644544 | 142 | A-1793284 | A-2093327 | rs10805643 |
| roh115 | 5 | p13.1 | p12 | 41477801 | 42878860 | 1401059 | 169 | A-2095298 | A-2121851 | rs318065 |
| roh116 | 5 | p12 | q11.1 | 43155707 | 50188224 | 7032517 | 243 | A-2119424 | A-4259195 | rs878234 |
| roh117 | 5 | q11.2 | q11.2 | 54359983 | 55286209 | 926226 | 112 | A-4228033 | A-2073730 | rs3776038 |
| roh118 | 5 | q12.1 | q12.1 | 59606386 | 60943787 | 1337401 | 188 | A-1838744 | A-1981827 | ? |
| roh119 | 5 | q13.3 | q13.3 | 74380221 | 75262237 | 882016 | 110 | A-4221696 | A-2260318 | rs6861101 |
| roh120 | 5 | q14.3 | q14.3 | 87136020 | 88110905 | 974885 | 109 | A-2063722 | A-2280527 | rs2032886 |
| roh121 | 5 | q21.1 | q21.1 | 101176858 | 102453650 | 1276792 | 184 | A-1941287 | A-2299109 | rs354663 |
| roh122 | 5 | q21.3 | q22.1 | 109461790 | 110527038 | 1065248 | 122 | A-2154936 | A-4237078 | rs1378509 |
| roh123 | 5 | q22.2 | q22.3 | 112050556 | 113229424 | 1178868 | 243 | A-4298952 | A-1982992 | rs1441368 |
| roh124 | 5 | q23.1 | q23.1 | 115398981 | 115820267 | 421286 | 124 | A-2281439 | A-2077019 | rs17138701 |
| roh125 | 5 | q23.3 | q23.3 | 129481382 | 132022568 | 2541186 | 286 | A-1983460 | A-1893772 | rs10491275 |
| roh126 | 5 | q31.1 | q31.1 | 135363750 | 135990446 | 626696 | 108 | A-4195063 | A-1818682 | rs17169651 |
| roh127 | 5 | q31.3 | q31.3 | 142011935 | 142509574 | 497639 | 119 | A-1796681 | A-1983781 | rs17099158 |
| roh128 | 5 | q32 | q32 | 147371778 | 147841128 | 469350 | 107 | A-2074593 | A-2212025 | rs10463395 |
| roh129 | 5 | q33.2 | q33.2 | 154592379 | 155033077 | 440698 | 116 | A-1872411 | A-4196159 | rs17653965 |
| roh130 | 5 | q33.3 | q33.3 | 155681482 | 156298759 | 617277 | 115 | A-2112079 | A-1984200 | rs10038955 |
| roh131 | 5 | q34 | q34 | 160450027 | 160944054 | 494027 | 103 | A-1904957 | A-1795630 | rs7734700 |
| roh132 | 6 | p24.3 | p24.3 | 8293905 | 8745764 | 451859 | 107 | A-1985004 | A-1900563 | rs7771933 |
| roh133 | 6 | p22.3 | p22.3 | 18511706 | 19369741 | 858035 | 202 | A-4202102 | A-1843690 | rs16880648 |
| roh134 | 6 | p22.2 | p22.1 | 26216147 | 29800284 | 3584137 | 489 | A-1869434 | A-2133768 | rs2051542 |
| roh135 | 6 | p21.33 | p21.33 | 30100240 | 30475206 | 374966 | 135 | A-2137088 | A-1985598 | rs1150743 |
| roh136 | 6 | p21.31 | p21.31 | 34544948 | 35579224 | 1034276 | 131 | A-1802082 | A-4245964 | rs3800473 |
| roh137 | 6 | p21.1 | p21.1 | 44714285 | 45506135 | 791850 | 116 | A-2065095 | A-2061705 | rs2153164 |
| roh138 | 6 | p12.3 | p12.3 | 48394735 | 49642247 | 1247512 | 172 | A-1819826 | A-4245122 | rs6902115 |
| roh139 | 6 | p12.2 | p12.2 | 51308502 | 51930366 | 621864 | 141 | A-2044095 | A-1986046 | rs6931113 |
| roh140 | 6 | p12.1 | p12.1 | 54011111 | 54983285 | 972174 | 177 | A-2026891 | A-2079341 | rs9637973 |
| roh141 | 6 | q11.1 | q12 | 62030184 | 65527416 | 3497232 | 424 | A-1986179 | A-4211696 | rs1689237 |
| roh142 | 6 | q12 | q13 | 69937498 | 70632669 | 695171 | 103 | A-4251849 | A-1905268 | rs7741707 |
| roh143 | 6 | q13 | q13 | 74636928 | 75405200 | 768272 | 137 | A-4193920 | A-4240524 | rs3012561 |
| roh144 | 6 | q14.1 | q14.1 | 80861133 | 82436294 | 1575161 | 256 | A-2169160 | A-2290573 | rs2505949 |
| roh145 | 6 | q14.3 | q14.3 | 85317123 | 85929713 | 612590 | 166 | A-2201442 | A-2277825 | rs6910673 |
| roh146 | 6 | q15 | q15 | 87546951 | 88573759 | 1026808 | 135 | A-2307945 | A-1921233 | rs9359715 |
| roh147 | 6 | q16.3 | q16.3 | 100927512 | 101967439 | 1039927 | 145 | A-4265840 | A-4221447 | rs17793103 |
| roh148 | 6 | q22.31 | q22.31 | 118601518 | 119172058 | 570540 | 105 | A-2050282 | A-2205732 | rs205946 |
| roh149 | 6 | q22.31 | q22.31 | 120403751 | 121805705 | 1401954 | 182 | A-1853940 | A-2039723 | rs9401205 |
| roh150 | 6 | q23.1 | q23.2 | 130527571 | 131420620 | 893049 | 194 | A-2210947 | A-2289948 | rs6569658 |
| roh151 | 6 | q24.2 | q24.3 | 145020197 | 146711619 | 1691422 | 239 | A-2123365 | A-1987997 | rs6570639 |
| roh152 | 6 | q27 | q27 | 169613057 | 170327598 | 714541 | 114 | A-2210480 | A-4283063 | rs418575 |
| roh153 | 7 | p14.3 | p14.3 | 32220323 | 33017621 | 797298 | 134 | A-2196498 | A-1989460 | rs384711 |
| roh154 | 7 | p12.2 | p12.2 | 50249685 | 50685887 | 436202 | 158 | A-2273846 | A-2246170 | rs4618649 |
| roh155 | 7 | p12.1 | p11.2 | 53437957 | 54480495 | 1042538 | 177 | A-4240076 | A-2101118 | rs6593088 |
| roh156 | 7 | q11.21 | q11.21 | 62762408 | 63464534 | 702126 | 113 | A-2134968 | A-2272956 | rs2019225 |
| roh157 | 7 | q11.22 | q11.22 | 68351912 | 69391615 | 1039703 | 111 | A-2186372 | A-4266408 | rs4718891 |
| roh158 | 7 | q21.11 | q21.11 | 83455840 | 84585437 | 1129597 | 132 | A-1833585 | A-2051827 | rs17158709 |
| roh159 | 7 | q21.11 | q21.12 | 85851481 | 86597050 | 745569 | 121 | A-2021938 | A-1861723 | rs12704267 |
| roh160 | 7 | q31.1 | q31.1 | 107238759 | 108305273 | 1066514 | 211 | A-4278112 | A-2105239 | rs3814474 |
| roh161 | 7 | q31.31 | q31.31 | 117380989 | 120202402 | 2821413 | 250 | A-1991015 | A-2254758 | rs6971712 |
| roh162 | 7 | q31.33 | q32.1 | 124813855 | 126162783 | 1348928 | 231 | A-2030115 | A-2149373 | rs1833082 |
| roh163 | 7 | q32.1 | q32.1 | 126463373 | 127377349 | 913976 | 150 | A-2030724 | A-1836205 | rs10226917 |
| roh164 | 7 | q33 | q33 | 132262359 | 133486536 | 1224177 | 203 | A-1806489 | A-2165480 | rs2343323 |
| roh165 | 8 | p23.1 | p23.1 | 8269203 | 8772623 | 503420 | 141 | A-1809943 | A-2144258 | rs4840946 |
| roh166 | 8 | p23.1 | p23.1 | 9207278 | 10144729 | 937451 | 242 | A-4296077 | A-4272491 | rs330101 |
| roh167 | 8 | p23.1 | p23.1 | 10567372 | 11074209 | 506837 | 121 | A-2094967 | A-2177718 | rs12543900 |
| roh168 | 8 | p22 | p22 | 15853794 | 16669543 | 815749 | 171 | A-1841108 | A-2062484 | rs17614179 |
| roh169 | 8 | p22 | p22 | 17625435 | 18020166 | 394731 | 137 | A-4297318 | A-2096133 | rs17634077 |
| roh170 | 8 | p21.2 | p21.2 | 23843704 | 24533615 | 689911 | 118 | A-1911371 | A-4266863 | rs17787051 |
| roh171 | 8 | p12 | p12 | 33590815 | 36749387 | 3158572 | 443 | A-1942156 | A-4280575 | rs7840674 |
| roh172 | 8 | p11.21 | q11.23 | 42588087 | 53273605 | 10685518 | 852 | A-2083540 | A-2122724 | rs2974317 |
| roh173 | 8 | q12.1 | q12.1 | 57989122 | 58616467 | 627345 | 120 | A-2179279 | A-2114683 | rs12681817 |
| roh174 | 8 | q12.1 | q12.1 | 60382882 | 61484269 | 1101387 | 146 | A-2070930 | A-2055352 | rs10957086 |
| roh175 | 8 | q12.3 | q12.3 | 63319367 | 64333676 | 1014309 | 151 | A-4235259 | A-1993580 | rs10957216 |
| roh176 | 8 | q21.11 | q21.12 | 78179199 | 78920541 | 741342 | 125 | A-1960804 | A-2258586 | rs16939419 |
| roh177 | 8 | q21.13 | q21.2 | 84014407 | 85141608 | 1127201 | 145 | A-2296805 | A-1903368 | rs2895873 |
| roh178 | 8 | q21.2 | q21.3 | 85313533 | 87124770 | 1811237 | 133 | A-2083464 | A-1916801 | rs10105026 |
| roh179 | 8 | q21.3 | q21.3 | 89279952 | 90163326 | 883374 | 131 | A-2199151 | A-2227745 | rs17666091 |
| roh180 | 8 | q21.3 | q21.3 | 90935078 | 91803658 | 868580 | 128 | A-1803383 | A-1994221 | rs432457 |
| roh181 | 8 | q23.1 | q23.1 | 107308956 | 108008872 | 699916 | 145 | A-2013698 | A-1876289 | rs6469053 |
| roh182 | 8 | q23.2 | q23.3 | 111369329 | 114842905 | 3473576 | 368 | A-2058422 | A-2074368 | rs16880571 |
| roh183 | 8 | q23.3 | q23.3 | 116722193 | 117486703 | 764510 | 103 | A-1906779 | A-2156294 | rs2178950 |
| roh184 | 8 | q24.12 | q24.12 | 120257010 | 120741833 | 484823 | 114 | A-2104896 | A-4192892 | rs2465373 |
| roh185 | 8 | q24.12 | q24.12 | 120761266 | 121292217 | 530951 | 109 | A-4246611 | A-1883118 | rs2082587 |
| roh186 | 8 | q24.21 | q24.21 | 129378665 | 129868152 | 489487 | 103 | A-4267234 | A-1820454 | rs16902791 |
| roh187 | 8 | q24.23 | q24.23 | 138362332 | 138646707 | 284375 | 102 | A-2013914 | A-1995382 | rs6577769 |
| roh188 | 9 | p24.3 | p24.3 | 463273 | 713773 | 250500 | 100 | A-2097707 | A-2087807 | rs3739595 |
| roh189 | 9 | p24.3 | p24.3 | 1486606 | 1823110 | 336504 | 165 | A-4205433 | A-4223976 | rs7850035 |
| roh190 | 9 | p23 | p23 | 11356989 | 11809477 | 452488 | 100 | A-4245069 | A-2140031 | rs10959745 |
| roh191 | 9 | p22.3 | p22.3 | 15360195 | 16096659 | 736464 | 139 | A-2153789 | A-2059097 | rs1343438 |
| roh192 | 9 | p21.2 | p21.2 | 25978943 | 26501000 | 522057 | 110 | A-2041166 | A-4226985 | rs2778457 |
| roh193 | 9 | p21.1 | p21.1 | 28642515 | 29259540 | 617025 | 158 | A-1996424 | A-1933778 | rs1438472 |
| roh194 | 9 | p21.1 | p21.1 | 29924085 | 30928018 | 1003933 | 164 | A-1959552 | A-4213469 | rs10969456 |
| roh195 | 9 | p21.1 | p21.1 | 31752133 | 32248150 | 496017 | 121 | A-2082822 | A-1855833 | rs706096 |
| roh196 | 9 | q21.13 | q21.13 | 72087672 | 72721268 | 633596 | 114 | A-2031177 | A-2188829 | rs4130866 |
| roh197 | 9 | q21.13 | q21.13 | 73443296 | 74180922 | 737626 | 107 | A-1813492 | A-1796558 | ? |
| roh198 | 9 | q22.31 | q22.31 | 91595317 | 92647665 | 1052348 | 146 | A-1816287 | A-1997234 | rs10992082 |
| roh199 | 9 | q22.32 | q22.32 | 94378610 | 95234370 | 855760 | 149 | A-2157181 | A-2287164 | rs10993232 |
| roh200 | 9 | q31.1 | q31.2 | 105034247 | 105820216 | 785969 | 133 | A-4243761 | A-1923089 | rs10991581 |
| roh201 | 9 | q33.2 | q33.2 | 120252072 | 121423254 | 1171182 | 179 | A-1795057 | A-1932781 | rs16909765 |
| roh202 | 9 | q33.3 | q33.3 | 124891685 | 125863535 | 971850 | 133 | A-1860818 | A-2275751 | ? |
| roh203 | 10 | p13 | p13 | 15561885 | 15881059 | 319174 | 104 | A-1955591 | A-1850421 | rs17369299 |
| roh204 | 10 | p12.31 | p12.2 | 22060671 | 23375963 | 1315292 | 169 | A-4229821 | A-1821061 | rs10430587 |
| roh205 | 10 | p12.2 | p12.2 | 23705444 | 24269452 | 564008 | 137 | A-4267790 | A-1925033 | rs1398024 |
| roh206 | 10 | p12.1 | p12.1 | 26227723 | 26678891 | 451168 | 108 | A-2041707 | A-2108299 | rs4749082 |
| roh207 | 10 | p11.22 | p11.22 | 32592460 | 33480185 | 887725 | 141 | A-4244243 | A-4286971 | rs7915416 |
| roh208 | 10 | p11.21 | p11.21 | 37565628 | 38663381 | 1097753 | 172 | A-1944208 | A-4214026 | rs2765819 |
| roh209 | 10 | q11.21 | q11.21 | 43623673 | 44206212 | 582539 | 193 | A-2168400 | A-2135374 | rs17463850 |
| roh210 | 10 | q11.23 | q11.23 | 49849207 | 50250881 | 401674 | 110 | A-4272283 | A-2247066 | rs17009410 |
| roh211 | 10 | q11.23 | q11.23 | 51777126 | 52116588 | 339462 | 103 | A-2022957 | A-4279766 | rs11005290 |
| roh212 | 10 | q21.1 | q21.1 | 55021305 | 55536088 | 514783 | 108 | A-1859270 | A-4239341 | rs10825067 |
| roh213 | 10 | q21.1 | q21.1 | 57174588 | 58993694 | 1819106 | 369 | A-4172771 | A-1788385 | rs10509041 |
| roh214 | 10 | q21.1 | q21.1 | 59103611 | 59622636 | 519025 | 122 | A-2221174 | A-1907862 | rs11815799 |
| roh215 | 10 | q21.1 | q21.2 | 60463422 | 61076392 | 612970 | 140 | A-4281476 | A-4247655 | rs11006353 |
| roh216 | 10 | q21.2 | q21.2 | 61680529 | 62170606 | 490077 | 104 | A-1947918 | A-1820900 | rs12767186 |
| roh217 | 10 | q21.3 | q21.3 | 64979873 | 66045964 | 1066091 | 188 | A-2038226 | A-2034732 | rs12761962 |
| roh218 | 10 | q21.3 | q21.3 | 68416291 | 69720623 | 1304332 | 170 | A-4272191 | A-2282450 | rs10997469 |
| roh219 | 10 | q22.1 | q22.2 | 73379996 | 75302766 | 1922770 | 150 | A-1897904 | A-2176677 | rs10762497 |
| roh220 | 10 | q22.3 | q22.3 | 77781626 | 78370108 | 588482 | 149 | A-2277731 | A-2093036 | rs7920287 |
| roh221 | 10 | q23.1 | q23.1 | 84413144 | 85448683 | 1035539 | 273 | A-2003306 | A-4214280 | rs17100439 |
| roh222 | 10 | q23.1 | q23.2 | 86447989 | 87242811 | 794822 | 206 | A-2113905 | A-2026152 | rs12416561 |
| roh223 | 10 | q24.2 | q24.2 | 100219496 | 100992214 | 772718 | 153 | A-2234979 | A-4193277 | rs1998757 |
| roh224 | 10 | q24.33 | q25.1 | 105634726 | 106217823 | 583097 | 133 | A-4251369 | A-2089009 | rs4918068 |
| roh225 | 10 | q25.1 | q25.1 | 106395925 | 107663097 | 1267172 | 308 | A-2206159 | A-4225273 | rs1413714 |
| roh226 | 10 | q25.1 | q25.1 | 108087508 | 108623596 | 536088 | 115 | A-1943203 | A-2081598 | rs17246894 |
| roh227 | 10 | q25.1 | q25.1 | 108879710 | 109325645 | 445935 | 113 | A-4214073 | A-4200017 | rs661162 |
| roh228 | 10 | q25.1 | q25.1 | 109733225 | 110327777 | 594552 | 140 | A-1855563 | A-4286497 | rs10787064 |
| roh229 | 10 | q25.1 | q25.1 | 110692416 | 111220147 | 527731 | 123 | A-2122263 | A-2212336 | rs11596345 |
| roh230 | 10 | q25.2 | q25.2 | 112918026 | 113940408 | 1022382 | 286 | A-2059466 | A-2001435 | rs11195449 |
| roh231 | 10 | q25.3 | q25.3 | 116561786 | 117695512 | 1133726 | 157 | A-2164061 | A-2005714 | rs11196924 |
| roh232 | 10 | q26.12 | q26.12 | 121726689 | 121996946 | 270257 | 102 | A-1881924 | A-2033080 | rs3999564 |
| roh233 | 10 | q26.12 | q26.12 | 122816800 | 123034204 | 217404 | 102 | A-1805032 | A-1871127 | rs7474750 |
| roh234 | 11 | p14.3 | p14.3 | 25212536 | 25792466 | 579930 | 129 | A-2008801 | A-2008880 | rs4584535 |
| roh235 | 11 | p14.1 | p14.1 | 28585691 | 29165952 | 580261 | 101 | A-1937402 | A-2009200 | rs11030385 |
| roh236 | 11 | p14.1 | p13 | 30697796 | 31892141 | 1194345 | 159 | A-2222526 | A-4296984 | rs7931310 |
| roh237 | 11 | p13 | p13 | 34623426 | 35041062 | 417636 | 149 | A-4230856 | A-2024264 | rs3751076 |
| roh238 | 11 | p12 | p12 | 37492528 | 40090659 | 2598131 | 386 | A-2054235 | A-4269519 | rs11034183 |
| roh239 | 11 | p12 | p12 | 41699048 | 42392725 | 693677 | 146 | A-2141035 | A-2010575 | rs479434 |
| roh240 | 11 | p11.2 | p11.2 | 45088245 | 45536675 | 448430 | 108 | A-2235086 | A-1896951 | rs1318730 |
| roh241 | 11 | p11.2 | p11.12 | 46212732 | 49874378 | 3661646 | 323 | A-4257606 | A-2010962 | rs7104451 |
| roh242 | 11 | p11.12 | q11 | 50407260 | 56412326 | 6005066 | 332 | A-2184782 | A-4273110 | rs11245687 |
| roh243 | 11 | q11 | q12.1 | 56481201 | 58181800 | 1700599 | 301 | A-1807686 | A-2170904 | rs1793447 |
| roh244 | 11 | q12.1 | q12.1 | 58602767 | 59583253 | 980486 | 150 | A-1950615 | A-2001975 | rs11229788 |
| roh245 | 11 | q13.4 | q13.4 | 72987532 | 73803671 | 816139 | 100 | A-2306744 | A-2206085 | rs11235817 |
| roh246 | 11 | q14.2 | q14.3 | 87622650 | 88460249 | 837599 | 152 | A-4253659 | A-2159786 | rs17754282 |
| roh247 | 11 | q14.3 | q14.3 | 89599131 | 90702192 | 1103061 | 245 | A-2013366 | A-1781852 | rs10501715 |
| roh248 | 11 | q14.3 | q21 | 91664434 | 92201848 | 537414 | 121 | A-2055220 | A-2088851 | rs2212389 |
| roh249 | 11 | q21 | q21 | 93153885 | 93707471 | 553586 | 102 | A-1920252 | A-2045614 | rs584324 |
| roh250 | 11 | q22.3 | q22.3 | 102488778 | 102947117 | 458339 | 103 | A-1817526 | A-1856346 | rs2513998 |
| roh251 | 11 | q22.3 | q22.3 | 103532187 | 105750701 | 2218514 | 379 | A-4194486 | A-1879228 | rs1351736 |
| roh252 | 11 | q23.1 | q23.1 | 112247308 | 112629928 | 382620 | 101 | A-2094695 | A-2050253 | rs3851570 |
| roh253 | 11 | q24.1 | q24.2 | 123431316 | 123905898 | 474582 | 162 | A-2188562 | A-2120530 | rs1453630 |
| roh254 | 12 | p12.3 | p12.3 | 18787125 | 19491448 | 704323 | 100 | A-4285317 | A-1841981 | rs10841081 |
| roh255 | 12 | p11.22 | p11.22 | 28133960 | 28636163 | 502203 | 100 | A-2044461 | A-1891570 | rs3912167 |
| roh256 | 12 | p11.22 | p11.22 | 30247605 | 30561782 | 314177 | 122 | A-2117313 | A-2053629 | rs11836490 |
| roh257 | 12 | p11.1 | p11.1 | 33290320 | 34219171 | 928851 | 121 | A-2028776 | A-2166704 | rs4244864 |
| roh258 | 12 | q11 | q12 | 36248035 | 37882928 | 1634893 | 181 | A-1862771 | A-1857170 | rs11168334 |
| roh259 | 12 | q12 | q12 | 39489064 | 40314746 | 825682 | 174 | A-2052230 | A-2268596 | rs984129 |
| roh260 | 12 | q12 | q12 | 42413091 | 43236324 | 823233 | 116 | A-2105173 | A-2218408 | rs11182236 |
| roh261 | 12 | q12 | q12 | 43521243 | 44228635 | 707392 | 100 | A-4194553 | A-4202005 | rs11182710 |
| roh262 | 12 | q13.11 | q13.11 | 46655256 | 47279215 | 623959 | 101 | A-2263670 | A-2209757 | rs4760608 |
| roh263 | 12 | q13.13 | q13.13 | 50977370 | 51216529 | 239159 | 114 | A-2059227 | A-4250186 | rs949387 |
| roh264 | 12 | q14.1 | q14.1 | 57434906 | 57920752 | 485846 | 116 | A-1883150 | A-4242576 | rs2605422 |
| roh265 | 12 | q14.1 | q14.1 | 58392780 | 59127069 | 734289 | 130 | A-2312969 | A-2065266 | rs11173119 |
| roh266 | 12 | q14.3 | q15 | 65715007 | 66154972 | 439965 | 103 | A-2065969 | A-2144316 | rs17836619 |
| roh267 | 12 | q21.1 | q21.1 | 72069387 | 73343253 | 1273866 | 160 | A-2158092 | A-4203496 | rs1520550 |
| roh268 | 12 | q21.2 | q21.31 | 77958388 | 79449679 | 1491291 | 207 | A-2187104 | A-2002885 | rs7971081 |
| roh269 | 12 | q21.31 | q21.31 | 79689761 | 80621273 | 931512 | 134 | A-1875000 | A-1924660 | rs17007357 |
| roh270 | 12 | q21.31 | q21.31 | 82360242 | 83585929 | 1225687 | 219 | A-2250485 | A-4253584 | rs17010860 |
| roh271 | 12 | q21.31 | q21.32 | 83871721 | 85955330 | 2083609 | 219 | A-1950526 | A-4273447 | rs1525547 |
| roh272 | 12 | q21.32 | q21.33 | 86743013 | 87983028 | 1240015 | 164 | A-4221284 | A-2111219 | rs7959909 |
| roh273 | 12 | q21.33 | q21.33 | 89795732 | 90539887 | 744155 | 144 | A-2147181 | A-2288842 | rs7131940 |
| roh274 | 12 | q23.1 | q23.1 | 95324037 | 95896606 | 572569 | 117 | A-1954555 | A-2089389 | rs7972539 |
| roh275 | 12 | q24.11 | q24.13 | 109752647 | 111733445 | 1980798 | 205 | A-2090087 | A-4282530 | rs16941053 |
| roh276 | 12 | q24.21 | q24.21 | 112817793 | 113047186 | 229393 | 103 | A-1793088 | A-4255277 | rs4767163 |
| roh277 | 13 | q14.11 | q14.11 | 42799471 | 43477797 | 678326 | 172 | A-1826150 | A-2045615 | rs4941455 |
| roh278 | 13 | q14.3 | q14.3 | 52544727 | 53432183 | 887456 | 144 | A-4258191 | A-2150978 | rs2799341 |
| roh279 | 13 | q21.1 | q21.1 | 54371695 | 56035097 | 1663402 | 215 | A-1816676 | A-1956848 | rs1350912 |
| roh280 | 13 | q21.1 | q21.1 | 56761736 | 58074616 | 1312880 | 153 | A-2026948 | A-2239295 | rs4886012 |
| roh281 | 13 | q21.2 | q21.31 | 61040040 | 61859645 | 819605 | 112 | A-2129366 | A-2191389 | rs4496012 |
| roh282 | 13 | q21.33 | q21.33 | 71340310 | 71792838 | 452528 | 124 | A-2165495 | A-1847220 | rs1325341 |
| roh283 | 13 | q31.2 | q31.3 | 87858739 | 88946115 | 1087376 | 147 | A-4287075 | A-4238244 | rs397548 |
| roh284 | 13 | q32.1 | q32.1 | 95145178 | 96275492 | 1130314 | 118 | A-4248664 | A-1887004 | rs9561932 |
| roh285 | 14 | q13.3 | q21.1 | 36532598 | 37318961 | 786363 | 104 | A-4232370 | A-1888308 | rs911008 |
| roh286 | 14 | q22.2 | q22.3 | 54327340 | 54985978 | 658638 | 102 | A-2208975 | A-4205775 | rs3813402 |
| roh287 | 14 | q23.1 | q23.1 | 58913159 | 60722080 | 1808921 | 232 | A-2111423 | A-1813139 | rs1253192 |
| roh288 | 14 | q23.1 | q23.2 | 60762952 | 61436288 | 673336 | 136 | A-1917810 | A-2193005 | rs17098148 |
| roh289 | 14 | q23.2 | q23.2 | 62812963 | 63683226 | 870263 | 100 | A-4291260 | A-2085231 | rs7141481 |
| roh290 | 14 | q23.3 | q23.3 | 64131739 | 64668505 | 536766 | 113 | A-2028809 | A-2225998 | rs971736 |
| roh291 | 14 | q23.3 | q24.1 | 65475183 | 67065410 | 1590227 | 197 | A-1935305 | A-1812845 | rs4902439 |
| roh292 | 14 | q24.1 | q24.1 | 67116492 | 67948844 | 832352 | 147 | A-1792986 | A-2227971 | rs2319843 |
| roh293 | 14 | q32.2 | q32.2 | 97421430 | 97833003 | 411573 | 124 | A-4201713 | A-2194713 | rs1379228 |
| roh294 | 15 | q14 | q15.1 | 37474419 | 37924307 | 449888 | 109 | A-2305746 | A-2273042 | rs8043207 |
| roh295 | 15 | q15.1 | q21.1 | 40233690 | 43748189 | 3514499 | 343 | A-1781290 | A-4280421 | rs677845 |
| roh296 | 15 | q21.1 | q21.1 | 45938882 | 46999340 | 1060458 | 208 | A-4224108 | A-1884806 | rs16960243 |
| roh297 | 15 | q21.2 | q21.2 | 48693072 | 49422190 | 729118 | 106 | A-2260179 | A-2041193 | rs511736 |
| roh298 | 15 | q21.2 | q21.3 | 49798782 | 50776788 | 978006 | 166 | A-4240134 | A-4236887 | rs16964486 |
| roh299 | 15 | q22.33 | q23 | 65195295 | 66039856 | 844561 | 131 | A-4236649 | A-2175652 | rs7168228 |
| roh300 | 15 | q23 | q23 | 66887071 | 67773628 | 886557 | 122 | A-2103857 | A-1799725 | rs2924631 |
| roh301 | 15 | q23 | q24.1 | 69711264 | 71611213 | 1899949 | 204 | A-1817396 | A-4239783 | rs4776565 |
| roh302 | 15 | q24.2 | q24.3 | 74271885 | 75256074 | 984189 | 100 | A-1935848 | A-2258313 | rs2456047 |
| roh303 | 15 | q25.3 | q25.3 | 84458029 | 84921859 | 463830 | 116 | A-1940590 | A-2091413 | rs4887287 |
| roh304 | 16 | p13.2 | p13.2 | 7165329 | 7381345 | 216016 | 104 | A-4201044 | A-4202495 | rs4411516 |
| roh305 | 16 | p13.2 | p13.2 | 10035456 | 10237758 | 202302 | 114 | A-1784960 | A-4192840 | rs3743833 |
| roh306 | 16 | p11.2 | q12.1 | 31511273 | 47118442 | 15607169 | 255 | A-2004667 | A-1807123 | rs4479245 |
| roh307 | 16 | q21 | q21 | 62772585 | 63454604 | 682019 | 133 | A-2069508 | A-2103489 | rs16967137 |
| roh308 | 16 | q22.1 | q22.1 | 65013143 | 67398089 | 2384946 | 209 | A-2042169 | A-2072861 | ? |
| roh309 | 16 | q23.1 | q23.1 | 75685094 | 75978734 | 293640 | 105 | A-1786889 | A-1862197 | rs607521 |
| roh310 | 16 | q23.1 | q23.1 | 76904717 | 77251169 | 346452 | 153 | A-2075480 | A-4218375 | rs4887952 |
| roh311 | 16 | q23.1 | q23.2 | 78255590 | 78825821 | 570231 | 237 | A-1841551 | A-4291996 | rs2348182 |
| roh312 | 16 | q23.2 | q23.3 | 80588235 | 80847224 | 258989 | 116 | A-1824507 | A-2160121 | rs16956155 |
| roh313 | 17 | q21.2 | q21.2 | 36289099 | 36964086 | 674987 | 171 | A-1872661 | A-2204634 | rs16966376 |
| roh314 | 17 | q21.31 | q21.32 | 41169023 | 42622984 | 1453961 | 211 | A-2313232 | A-2132974 | rs17563787 |
| roh315 | 17 | q22 | q22 | 47687556 | 48318148 | 630592 | 122 | A-1859483 | A-2100977 | rs1825734 |
| roh316 | 17 | q23.1 | q23.2 | 50950379 | 51522852 | 572473 | 145 | A-2103972 | A-2313192 | rs11079179 |
| roh317 | 17 | q23.2 | q23.2 | 54856683 | 56726816 | 1870133 | 144 | A-1872767 | A-4242245 | rs2012146 |
| roh318 | 18 | q11.2 | q11.2 | 20390749 | 20698252 | 307503 | 101 | A-2282609 | A-2052273 | rs7227781 |
| roh319 | 18 | q12.1 | q12.1 | 24241525 | 25138544 | 897019 | 118 | A-2226921 | A-2274232 | rs2140332 |
| roh320 | 18 | q12.1 | q12.1 | 28338083 | 29674646 | 1336563 | 218 | A-2048929 | A-2281833 | rs12454779 |
| roh321 | 18 | q12.3 | q12.3 | 37022928 | 37619977 | 597049 | 119 | A-2122598 | A-4248616 | rs2552480 |
| roh322 | 18 | q12.3 | q12.3 | 37974546 | 39207131 | 1232585 | 228 | A-4241770 | A-2005622 | rs7407986 |
| roh323 | 18 | q21.1 | q21.2 | 48479996 | 49338734 | 858738 | 170 | A-1912845 | A-4204303 | rs17749451 |
| roh324 | 18 | q21.2 | q21.2 | 49588927 | 50699931 | 1111004 | 207 | A-2005667 | A-4202796 | rs8091889 |
| roh325 | 18 | q21.33 | q21.33 | 57733482 | 58183929 | 450447 | 102 | A-2221346 | A-1921373 | rs948783 |
| roh326 | 19 | p12 | p12 | 20716764 | 21543729 | 826965 | 106 | A-4232594 | A-4194806 | ? |
| roh327 | 19 | p12 | q12 | 23174830 | 33154583 | 9979753 | 232 | A-2032460 | A-1861465 | rs10424248 |
| roh328 | 19 | q12 | 413.11 | 36675192 | 37131231 | 456039 | 106 | A-1887252 | A-2030267 | rs8111575 |
| roh329 | 19 | q13.12 | q13.13 | 41956030 | 43165900 | 1209870 | 100 | A-1780870 | A-1918904 | rs1673087 |
| roh330 | 20 | p11.23 | p11.21 | 21004822 | 22775321 | 1770499 | 335 | A-2272756 | A-2266327 | rs6035773 |
| roh331 | 20 | p11.21 | p11.21 | 23853650 | 24263129 | 409479 | 107 | A-4196606 | A-2275978 | rs1741632 |
| roh332 | 20 | q11.22 | q11.23 | 31977722 | 34401989 | 2424267 | 264 | A-1875231 | A-2038691 | rs6142046 |
| roh333 | 20 | q12 | q12 | 37132667 | 37920663 | 787996 | 185 | A-2090591 | A-2196736 | rs873082 |
| roh334 | 20 | q13.12 | q13.12 | 43495258 | 44044223 | 548965 | 125 | A-2062029 | A-1868863 | rs6032189 |
| roh335 | 20 | q13.2 | q13.2 | 52872796 | 53307708 | 434912 | 119 | A-2247443 | A-2216269 | rs2668396 |
| roh336 | 21 | q21.2 | q21.3 | 25306451 | 26125668 | 819217 | 122 | A-4269226 | A-4270304 | rs4550741 |
| roh337 | 21 | q21.3 | q22.11 | 29199538 | 30521327 | 1321789 | 259 | A-1969847 | A-2017806 | rs2248708 |
| roh338 | 22 | q12.1 | q12.2 | 26554620 | 28931481 | 2376861 | 297 | A-4302541 | A-2240075 | rs16985600 |
| roh339 | 22 | q12.2 | q12.3 | 29851365 | 30990000 | 1138635 | 157 | A-1819087 | A-2217887 | rs5753469 |
| roh340 | 22 | q13.1 | q13.2 | 39038116 | 41012088 | 1973972 | 180 | A-4290846 | A-2194547 | rs17001819 |

| ROH | End_rs | N_Cases | %_Cases | N_Cntl | % Cntl | Chi-sq | P | iHS_max | TajimaD_max | Fst_max |
|---|---|---|---|---|---|---|---|---|---|---|
| roh2 | rs4949455 | 10 | 5.6 | 4 | 2.8 | f | 0.276 | 1.00 | 0.10 | 0.45 |
| roh3 | rs4652914 | 15 | 8.4 | 12 | 8.3 | 0.001 | 0.976 | 3.70 | 3.70 | 0.55 |
| roh4 | rs3845301 | 12 | 6.7 | 10 | 6.9 | 0.005 | 0.943 | 0.60 | 0.70 | 0.15 |
| roh5 | rs2938100 | 20 | 11.2 | 23 | 16.0 | 1.543 | 0.214 | 0.80 | 1.70 | 0.68 |
| roh6 | rs10493208 | 29 | 16.3 | 13 | 9.0 | 3.704 | 0.054 | 2.20 | 1.50 | 0.30 |
| roh7 | rs1909067 | 23 | 12.9 | 27 | 18.8 | 2.062 | 0.151 | 1.20 | 1.00 | 0.45 |
| roh8 | rs11162069 | 6 | 3.4 | 14 | 9.7 | f | 0.021 | 0.00 | 0.70 | 0.20 |
| roh9 | rs11163019 | 7 | 3.9 | 7 | 4.9 | f | 0.786 | 1.00 | 0.70 | 0.30 |
| roh10 | rs12085660 | 7 | 3.9 | 8 | 5.6 | f | 0.598 | 1.50 | 1.60 | 0.12 |
| roh11 | rs290828 | 12 | 6.7 | 7 | 4.9 | f | 0.635 | 1.70 | 1.20 | 0.09 |
| roh12 | rs3181089 | 11 | 6.2 | 5 | 3.5 | f | 0.311 | 1.50 | 1.40 | 0.80 |
| roh13 | rs10493999 | 13 | 7.3 | 9 | 6.3 | f | 0.825 | 1.40 | 2.20 | 0.35 |
| roh14 | rs16835083 | 21 | 11.8 | 11 | 7.6 | 1.538 | 0.215 | 0.50 | 1.10 | 0.30 |
| roh15 | rs2819327 | 20 | 11.2 | 4 | 2.8 | 8.256 | 0.004 | 0.50 | 1.40 | 0.50 |
| roh16 | rs1387353 | 11 | 6.2 | 1 | 0.7 | f | 0.014 | 0.00 | 0.50 | 0.20 |
| roh17 | rs6673720 | 8 | 4.5 | 6 | 4.2 | f | 1.000 | 0.25 | 1.00 | 0.45 |
| roh18 | rs471569 | 16 | 9.0 | 4 | 2.8 | f | 0.034 | 0.50 | 1.00 | 0.20 |
| roh19 | rs16847820 | 22 | 12.4 | 18 | 12.5 | 0.001 | 0.970 | 1.50 | 1.00 | 0.58 |
| roh20 | rs7416836 | 14 | 7.9 | 12 | 8.3 | 0.024 | 0.878 | 1.50 | 1.70 | 0.35 |
| roh21 | rs16861188 | 10 | 5.6 | 9 | 6.3 | f | 0.817 | 1.60 | 2.00 | 0.48 |
| roh22 | rs13375677 | 11 | 6.2 | 8 | 5.6 | f | 1.000 | 1.50 | 0.50 | 0.20 |
| roh23 | rs1730733 | 16 | 9.0 | 9 | 6.3 | 0.834 | 0.361 | 1.60 | 1.60 | 0.20 |
| roh24 | rs1370881 | 13 | 7.3 | 5 | 3.5 | f | 0.152 | 0.60 | 0.70 | 0.35 |
| roh25 | rs10921643 | 12 | 6.7 | 9 | 6.3 | f | 1.000 | 1.70 | 0.50 | 0.12 |
| roh26 | rs2808019 | 15 | 8.4 | 3 | 2.1 | f | 0.014 | 2.00 | 1.50 | 0.48 |
| roh27 | rs10495234 | 15 | 8.4 | 6 | 4.2 | f | 0.173 | 0.25 | 1.00 | 0.35 |
| roh28 | rs3820573 | 16 | 9.0 | 4 | 2.8 | f | 0.034 | 0.60 | 0.25 | 0.15 |
| roh29 | rs4832500 | 12 | 6.7 | 6 | 4.2 | f | 0.343 | 0.25 | 1.40 | 0.10 |
| roh30 | rs4302239 | 16 | 9.0 | 8 | 5.6 | 1.36 | 0.243 | 0.00 | 3.70 | 0.25 |
| roh31 | rs7603886 | 14 | 7.9 | 11 | 7.6 | 0.006 | 0.940 | 1.20 | 2.20 | 0.65 |
| roh32 | rs410259 | 11 | 6.2 | 8 | 5.6 | f | 1.000 | 0.50 | 1.40 | 0.55 |
| roh33 | rs6738287 | 8 | 4.5 | 8 | 5.6 | f | 0.798 | 0.25 | 0.20 | 0.25 |
| roh34 | rs13414722 | 10 | 5.6 | 7 | 4.9 | f | 0.808 | 0.80 | 0.40 | 0.25 |
| roh35 | rs9789714 | 4 | 2.2 | 11 | 7.6 | f | 0.031 | 0.75 | 1.00 | 0.42 |
| roh36 | rs7606191 | 9 | 5.1 | 5 | 3.5 | f | 0.589 | 2.70 | 0.50 | 0.00 |
| roh37 | rs736711 | 45 | 25.3 | 29 | 20.1 | 1.189 | 0.276 | 1.50 | 2.25 | 0.20 |
| roh38 | rs6547701 | 17 | 9.6 | 10 | 6.9 | 0.704 | 0.402 | 0.50 | 1.10 | 0.30 |
| roh39 | rs6708290 | 13 | 7.3 | 10 | 6.9 | 0.015 | 0.901 | 0.55 | 0.50 | 0.15 |
| roh40 | rs17048379 | 9 | 5.1 | 11 | 7.6 | f | 0.362 | 1.20 | 1.00 | 2.10 |
| roh41 | rs6728856 | 22 | 12.4 | 12 | 8.3 | 1.366 | 0.242 | 1.25 | 1.20 | 0.57 |
| roh42 | rs17821815 | 27 | 15.2 | 31 | 21.5 | 2.18 | 0.140 | 1.20 | 1.50 | 0.60 |
| roh43 | rs13385327 | 8 | 4.5 | 7 | 4.9 | f | 1.000 | 0.75 | 0.70 | 0.10 |
| roh44 | rs6431110 | 18 | 10.1 | 12 | 8.3 | 0.298 | 0.585 | 0.75 | 0.25 | 0.50 |
| roh45 | rs1432231 | 24 | 13.5 | 15 | 10.4 | 0.703 | 0.402 | 4.50 | 2.20 | 0.38 |
| roh46 | rs1370504 | 22 | 12.4 | 19 | 13.2 | 0.05 | 0.823 | 2.25 | 1.50 | 0.42 |
| roh47 | rs16842296 | 8 | 4.5 | 7 | 4.9 | f | 1.000 | 1.50 | 2.50 | 0.72 |
| roh48 | rs2528632 | 8 | 4.5 | 9 | 6.3 | f | 0.618 | 2.25 | 1.40 | 0.60 |
| roh49 | rs7594813 | 13 | 7.3 | 14 | 9.7 | 0.606 | 0.436 | 1.50 | 1.75 | 0.45 |
| roh50 | rs11900934 | 9 | 5.1 | 8 | 5.6 | f | 1.000 | 0.25 | 3.80 | 0.58 |
| roh51 | rs16853735 | 8 | 4.5 | 8 | 5.6 | f | 0.798 | 2.10 | 1.70 | 0.40 |
| roh52 | rs16863378 | 17 | 9.6 | 2 | 1.4 | f | 0.002 | 2.60 | 1.50 | 0.28 |
| roh53 | rs4638831 | 26 | 14.6 | 29 | 20.1 | 1.72 | 0.190 | 2.25 | 1.25 | 0.40 |
| roh54 | rs10203398 | 26 | 14.6 | 12 | 8.3 | 3.01 | 0.083 | 1.20 | 1.25 | 0.55 |
| roh55 | rs785244 | 31 | 17.4 | 10 | 6.9 | 7.855 | 0.005 | 2.20 | 1.50 | 0.30 |
| roh56 | rs9283513 | 51 | 28.7 | 35 | 24.3 | 0.768 | 0.381 | 2.50 | 2.00 | 0.45 |
| roh57 | rs10211241 | 15 | 8.4 | 13 | 9.0 | 0.036 | 0.849 | 2.40 | 0.70 | 0.70 |
| roh58 | rs2887881 | 10 | 5.6 | 7 | 4.9 | f | 0.808 | 0.75 | 2.00 | 0.42 |
| roh59 | rs2373146 | 9 | 5.1 | 8 | 5.6 | f | 1.000 | 1.20 | 1.00 | 0.52 |
| roh60 | rs7596956 | 9 | 5.1 | 6 | 4.2 | f | 0.795 | 1.20 | 1.00 | 0.52 |
| roh61 | rs6734283 | 6 | 3.4 | 11 | 7.6 | f | 0.131 | 0.60 | 2.00 | 2.20 |
| roh62 | rs17039757 | 11 | 6.2 | 6 | 4.2 | f | 0.464 | 1.00 | 1.10 | 0.25 |
| roh63 | rs10865897 | 15 | 8.4 | 7 | 4.9 | f | 0.268 | 1.00 | 2.80 | 0.30 |
| roh64 | rs9852262 | 13 | 7.3 | 9 | 6.3 | f | 0.825 | 0.25 | 1.40 | 0.40 |
| roh65 | rs17075993 | 19 | 10.7 | 9 | 6.3 | 1.962 | 0.161 | 0.00 | 1.20 | 0.30 |
| roh66 | rs6441970 | 12 | 6.7 | 5 | 3.5 | f | 0.219 | 0.25 | 0.50 | 0.30 |
| roh67 | rs876597 | 14 | 7.9 | 15 | 10.4 | 0.632 | 0.426 | 1.25 | 0.75 | 0.40 |
| roh68 | rs1560332 | 32 | 18.0 | 29 | 20.1 | 0.242 | 0.623 | 1.00 | 1.75 | 0.28 |
| roh69 | rs978979 | 8 | 4.5 | 5 | 3.5 | f | 0.779 | 1.20 | 1.00 | 0.10 |
| roh70 | rs17047115 | 5 | 2.8 | 9 | 6.3 | f | 0.171 | 0.80 | 1.00 | 0.10 |
| roh71 | rs6777604 | 12 | 6.7 | 10 | 6.9 | f | 1.000 | 1.70 | 0.70 | 0.00 |
| roh72 | rs17028592 | 9 | 5.1 | 5 | 3.5 | f | 0.589 | 1.75 | 1.20 | 0.20 |
| roh73 | rs6548732 | 19 | 10.7 | 13 | 9.0 | 0.241 | 0.623 | 0.70 | 1.50 | 0.42 |
| roh74 | rs2122355 | 13 | 7.3 | 9 | 6.3 | f | 0.825 | 0.50 | 1.00 | 0.10 |
| roh75 | rs9857944 | 13 | 7.3 | 6 | 4.2 | f | 0.342 | 0.25 | 1.00 | 0.22 |
| roh76 | rs4362744 | 14 | 7.9 | 11 | 7.6 | 0.006 | 0.940 | 0.25 | 0.75 | 0.85 |
| roh77 | rs1009521 | 36 | 20.2 | 28 | 19.4 | 0.03 | 0.862 | 1.25 | 2.70 | 0.57 |
| roh78 | rs17315194 | 11 | 6.2 | 13 | 9.0 | 0.936 | 0.333 | 1.40 | 1.20 | 0.60 |
| roh79 | rs17202754 | 20 | 11.2 | 16 | 11.1 | 0.001 | 0.972 | 1.10 | 2.40 | 0.80 |
| roh80 | rs1518337 | 10 | 5.6 | 9 | 6.3 | f | 0.817 | 2.10 | 1.40 | 0.58 |
| roh81 | rs16835267 | 21 | 11.8 | 11 | 7.6 | 1.538 | 0.215 | 2.80 | 2.30 | 0.82 |
| roh82 | rs6763412 | 12 | 6.7 | 5 | 3.5 | f | 0.219 | 1.00 | 0.20 | 0.15 |
| roh83 | rs1873521 | 12 | 6.7 | 8 | 5.6 | f | 0.817 | 1.10 | 0.70 | 0.00 |
| roh84 | rs4355238 | 15 | 8.4 | 3 | 2.1 | f | 0.014 | 2.00 | 0.70 | 0.35 |
| roh85 | rs6782299 | 11 | 6.2 | 14 | 9.7 | 1.395 | 0.238 | 1.30 | 1.40 | 0.52 |
| roh86 | rs6823189 | 8 | 4.5 | 4 | 2.8 | f | 0.558 | 1.60 | 2.00 | 0.50 |
| roh87 | rs6812714 | 22 | 12.4 | 21 | 14.6 | 0.34 | 0.560 | 1.60 | 1.40 | 0.30 |
| roh88 | rs 12502866 | 5 | 2.8 | 7 | 4.9 | f | 0.384 | 1.25 | 1.20 | 0.40 |
| roh89 | rs7654452 | 64 | 36.0 | 52 | 36.1 | 0.001 | 0.977 | 3.50 | 2.40 | 0.62 |
| roh90 | rs12502592 | 7 | 3.9 | 7 | 4.9 | f | 0.786 | 0.50 | 1.75 | 0.30 |
| roh91 | rs10805123 | 12 | 6.7 | 8 | 5.6 | f | 0.817 | 0.75 | 2.00 | 0.60 |
| roh92 | rs4865408 | 12 | 6.7 | 7 | 4.9 | f | 0.635 | 0.25 | 1.20 | 0.97 |
| roh93 | rs2124237 | 14 | 7.9 | 11 | 7.6 | 0.006 | 0.940 | 3.00 | 1.80 | 0.50 |
| roh94 | rs1376416 | 13 | 7.3 | 5 | 3.5 | f | 0.152 | 0.75 | 1.20 | 0.38 |
| roh95 | rs16846893 | 11 | 6.2 | 5 | 3.5 | f | 0.311 | 1.80 | 2.40 | 0.65 |
| roh96 | rs579866 | 17 | 9.6 | 5 | 3.5 | f | 0.044 | 0.60 | 1.50 | 0.50 |
| roh97 | rs1604153 | 26 | 14.6 | 22 | 15.3 | 0.028 | 0.867 | 0.00 | 0.70 | 0.18 |
| roh98 | rs13150370 | 12 | 6.7 | 12 | 8.3 | 0.292 | 0.589 | 1.00 | 2.75 | 0.70 |
| roh99 | ? | 12 | 6.7 | 12 | 8.3 | 0.292 | 0.589 | 2.75 | 1.60 | 0.58 |
| roh100 | rs10857061 | 12 | 6.7 | 6 | 4.2 | f | 0.343 | 2.00 | 1.50 | 0.50 |
| roh101 | rs6534156 | 16 | 9.0 | 6 | 4.2 | f | 0.119 | 0.25 | 0.60 | 0.00 |
| roh102 | rs10493148 | 15 | 8.4 | 16 | 11.1 | 0.659 | 0.417 | 0.70 | 0.80 | 0.30 |
| roh103 | rs1022006 | 11 | 6.2 | 2 | 1.4 | f | 0.043 | 0.00 | 0.50 | 0.55 |
| roh104 | rs12502464 | 26 | 14.6 | 20 | 13.9 | 0.033 | 0.855 | 1.50 | 1.75 | 0.60 |
| roh105 | rs6537474 | 13 | 7.3 | 13 | 9.0 | 0.319 | 0.572 | 2.25 | 1.80 | 0.22 |
| roh106 | rs7669465 | 8 | 4.5 | 8 | 5.6 | f | 0.798 | 1.50 | 3.00 | 0.60 |
| roh107 | rs9994520 | 8 | 4.5 | 7 | 4.9 | f | 1.000 | 0.60 | 0.70 | 0.40 |
| roh108 | rs4690909 | 11 | 6.2 | 6 | 4.2 | f | 0.464 | 0.70 | 1.50 | 0.50 |
| roh109 | rs1717072 | 13 | 7.3 | 6 | 4.2 | f | 0.342 | 0.60 | 0.60 | 0.25 |
| roh110 | rs17057275 | 14 | 7.9 | 13 | 9.0 | 0.14 | 0.708 | 1.20 | 1.25 | 0.60 |
| roh111 | rs10020676 | 9 | 5.1 | 5 | 3.5 | f | 0.589 | 0.75 | 0.80 | 0.38 |
| roh112 | rs4701970 | 10 | 5.6 | 7 | 4.9 | f | 0.808 | 0.60 | 1.00 | 0.35 |
| roh113 | rs12108871 | 13 | 7.3 | 10 | 6.9 | 0.015 | 0.901 | 1.75 | 1.50 | 0.35 |
| roh114 | rs351651 | 11 | 6.2 | 10 | 6.9 | f | 0.823 | 0.70 | 1.20 | 0.25 |
| roh115 | rs6882786 | 22 | 12.4 | 16 | 11.1 | 0.119 | 0.730 | 1.10 | 1.40 | 0.25 |
| roh116 | rs27964 | 19 | 10.7 | 18 | 12.5 | 0.261 | 0.609 | 1.10 | 2.10 | 0.10 |
| roh117 | rs6860698 | 12 | 6.7 | 12 | 8.3 | 0.292 | 0.589 | 2.30 | 1.80 | 0.25 |
| roh118 | rs895382 | 22 | 12.4 | 19 | 13.2 | 0.05 | 0.823 | 1.50 | 0.75 | 0.30 |
| roh119 | rs11960372 | 6 | 3.4 | 10 | 6.9 | f | 0.197 | 1.20 | 1.20 | 0.88 |
| roh120 | rs665369 | 7 | 3.9 | 9 | 6.3 | f | 0.441 | 1.40 | 2.25 | 0.45 |
| roh121 | rs34798 | 20 | 11.2 | 7 | 4.9 | 4.211 | 0.040 | 0.60 | 1.50 | 0.20 |
| roh122 | rs10054378 | 13 | 7.3 | 11 | 7.6 | 0.013 | 0.909 | 2.80 | 2.50 | 0.90 |
| roh123 | rs2035414 | 37 | 20.8 | 17 | 11.8 | 4.6 | 0.032 | 1.60 | 1.20 | 0.28 |
| roh124 | rs26604 | 14 | 7.9 | 13 | 9.0 | 0.14 | 0.708 | 0.80 | 0.80 | 0.70 |
| roh125 | rs2069744 | 46 | 25.8 | 36 | 25.0 | 0.03 | 0.863 | 1.20 | 1.25 | 0.65 |
| roh126 | rs11737955 | 11 | 6.2 | 6 | 4.2 | f | 0.464 | 0.50 | 0.30 | 0.25 |
| roh127 | rs1438733 | 14 | 7.9 | 21 | 14.6 | 3.708 | 0.054 | 2.00 | 2.25 | 0.65 |
| roh128 | rs2277051 | 14 | 7.9 | 5 | 3.5 | f | 0.152 | 0.00 | 1.25 | 0.55 |
| roh129 | rs6580176 | 13 | 7.3 | 1 | 0.7 | f | 0.004 | 1.20 | 0.60 | 0.25 |
| roh130 | rs7700944 | 10 | 5.6 | 7 | 4.9 | f | 0.808 | 0.25 | 1.60 | 0.10 |
| roh131 | rs4921399 | 8 | 4.5 | 3 | 2.1 | f | 0.357 | 0.75 | 0.80 | 0.15 |
| roh132 | rs4246080 | 16 | 9.0 | 5 | 3.5 | f | 0.067 | 0.40 | 0.75 | 0.12 |
| roh133 | rs10214554 | 13 | 7.3 | 12 | 8.3 | 0.118 | 0.731 | 1.20 | 0.80 | 0.12 |
| roh134 | rs1632953 | 51 | 28.7 | 52 | 36.1 | 2.036 | 0.154 | 2.40 | 1.50 | 0.52 |
| roh135 | ? | 11 | 6.2 | 10 | 6.9 | f | 0.823 | 2.00 | 0.70 | 0.00 |
| roh136 | rs2067997 | 17 | 9.6 | 11 | 7.6 | 0.366 | 0.545 | 3.00 | 1.70 | 0.62 |
| roh137 | rs2677101 | 15 | 8.4 | 10 | 6.9 | 0.244 | 0.621 | 0.00 | 0.75 | 0.08 |
| roh138 | rs6904033 | 19 | 10.7 | 19 | 13.2 | 0.486 | 0.486 | 0.60 | 0.70 | 0.25 |
| roh139 | rs1321506 | 11 | 6.2 | 7 | 4.9 | f | 0.636 | 1.40 | 1.30 | 0.18 |
| roh140 | rs6914527 | 20 | 14.4 | 6 | 4.2 | 5.36 | 0.021 | 0.70 | 1.20 | 0.32 |
| roh141 | rs10498827 | 35 | 19.7 | 31 | 21.5 | 0.17 | 0.680 | 1.20 | 1.30 | 0.15 |
| roh142 | rs1340960 | 8 | 4.5 | 4 | 2.8 | f | 0.558 | 1.40 | 1.10 | 0.10 |
| roh143 | rs6901402 | 16 | 9.0 | 7 | 4.9 | 2.045 | 0.153 | 1.30 | 1.70 | 0.15 |
| roh144 | rs1341230 | 27 | 15.2 | 18 | 12.5 | 0.472 | 0.492 | 0.40 | 1.00 | 0.28 |
| roh145 | rs581803 | 14 | 7.9 | 14 | 9.7 | 0.346 | 0.557 | 1.60 | 1.20 | 0.10 |
| roh146 | rs2787892 | 23 | 12.9 | 14 | 9.7 | 0.801 | 0.371 | 0.80 | 1.40 | 0.00 |
| roh147 | rs7754131 | 20 | 11.2 | 15 | 10.4 | 0.055 | 0.814 | 1.20 | 1.20 | 0.40 |
| roh148 | rs9385056 | 8 | 4.5 | 8 | 5.6 | f | 0.798 | 0.50 | 0.70 | 0.30 |
| roh149 | rs10456939 | 16 | 9.0 | 9 | 6.3 | 0.834 | 0.361 | 2.80 | 1.50 | 0.60 |
| roh150 | rs9398971 | 17 | 9.6 | 10 | 6.9 | 0.704 | 0.402 | 2.00 | 0.80 | 0.65 |
| roh151 | rs362868 | 18 | 10.1 | 16 | 11.1 | 0.084 | 0.772 | 1.70 | 3.10 | 0.62 |
| roh152 | rs6456234 | 12 | 6.7 | 7 | 4.9 | f | 0.635 | 1.80 | 1.10 | 0.32 |
| roh153 | rs6979640 | 10 | 5.6 | 8 | 5.6 | f | 1.000 | 0.80 | 0.60 | 0.30 |
| roh154 | rs10230084 | 10 | 5.6 | 7 | 4.9 | f | 0.808 | 0.40 | 0.75 | 0.65 |
| roh155 | ? | 18 | 10.1 | 6 | 4.2 | 4.08 | 0.043 | 0.90 | 0.80 | 0.20 |
| roh156 | rs7384477 | 10 | 5.6 | 6 | 4.2 | f | 0.614 | 0.25 | 1.00 | 0.09 |
| roh157 | rs10486873 | 12 | 6.7 | 5 | 3.5 | f | 0.219 | 2.60 | 1.25 | 0.09 |
| roh158 | rs2732778 | 14 | 7.9 | 8 | 5.6 | f | 0.508 | 0.70 | 0.80 | 0.18 |
| roh159 | rs1089468 | 14 | 7.9 | 5 | 3.5 | f | 0.152 | 0.60 | 1.00 | 0.38 |
| roh160 | rs7793335 | 16 | 9.0 | 12 | 8.3 | 0.043 | 0.836 | 1.00 | 2.50 | 0.58 |
| roh161 | rs17533951 | 40 | 22.5 | 25 | 17.4 | 1.291 | 0.256 | 1.70 | 2.70 | 0.60 |
| roh162 | rs3808142 | 20 | 11.2 | 9 | 6.3 | 2.415 | 0.120 | 1.70 | 1.00 | 0.22 |
| roh163 | rs322740 | 14 | 7.9 | 6 | 4.2 | f | 0.245 | 1.30 | 1.00 | 0.20 |
| roh164 | rs6972323 | 18 | 10.1 | 11 | 7.6 | 0.594 | 0.441 | 1.80 | 1.70 | 0.32 |
| roh165 | rs10046784 | 10 | 5.6 | 8 | 5.6 | f | 1.000 | 0.70 | 0.60 | 0.22 |
| roh166 | rs12544785 | 18 | 10.1 | 27 | 18.8 | 4.94 | 0.026 | 1.80 | 1.25 | 0.28 |
| roh167 | rs11777887 | 8 | 4.5 | 6 | 4.2 | f | 1.000 | 0.70 | 0.25 | 0.48 |
| roh168 | rs6586877 | 15 | 8.4 | 11 | 7.6 | 0.067 | 0.796 | 1.25 | 2.50 | 0.68 |
| roh169 | rs1029340 | 28 | 15.7 | 21 | 14.6 | 0.081 | 0.776 | 0.90 | 0.75 | 0.20 |
| roh170 | rs17052270 | 14 | 7.9 | 4 | 2.8 | f | 0.054 | 0.50 | 4.80 | 0.12 |
| roh171 | rs1981322 | 56 | 31.5 | 41 | 28.5 | 0.338 | 0.561 | 0.60 | 2.80 | 0.42 |
| roh172 | rs2360804 | 116 | 65.2 | 77 | 53.5 | 4.535 | 0.033 | 3.90 | 2.00 | 0.90 |
| roh173 | rs16922281 | 20 | 11.2 | 5 | 3.5 | 6.7 | 0.010 | 1.60 | 1.40 | 0.22 |
| roh174 | rs16926137 | 11 | 6.2 | 15 | 10.4 | 1.925 | 0.165 | 1.80 | 2.40 | 0.35 |
| roh175 | rs4739071 | 15 | 8.4 | 9 | 6.3 | 0.547 | 0.460 | 2.20 | 1.40 | 0.45 |
| roh176 | rs16076 | 9 | 5.1 | 6 | 4.2 | f | 0.795 | 0.80 | 1.25 | 0.15 |
| roh177 | rs16912447 | 13 | 7.3 | 9 | 6.3 | f | 0.825 | 1.80 | 2.20 | 0.48 |
| roh178 | ? | 11 | 6.2 | 11 | 7.6 | f | 0.660 | 0.80 | 1.15 | 0.20 |
| roh179 | rs1681444 | 13 | 7.3 | 4 | 2.8 | f | 0.083 | 1.80 | 1.10 | 0.42 |
| roh180 | rs2339228 | 15 | 8.4 | 8 | 5.6 | 0.99 | 0.320 | 1.70 | 1.50 | 0.40 |
| roh181 | rs16875331 | 12 | 6.7 | 6 | 4.2 | f | 0.343 | 0.75 | 0.80 | 0.60 |
| roh182 | rs3911267 | 26 | 14.6 | 23 | 16.0 | 0.115 | 0.734 | 2.25 | 1.80 | 0.52 |
| roh183 | rs6986302 | 8 | 4.5 | 6 | 4.2 | f | 1.000 | 0.00 | 1.25 | 0.52 |
| roh184 | rs16892921 | 9 | 5.1 | 8 | 5.6 | f | 1.000 | 2.40 | 1.50 | 0.20 |
| roh185 | rs11786178 | 11 | 6.2 | 12 | 8.3 | 0.557 | 0.456 | 2.25 | 1.75 | 0.09 |
| roh186 | rs1519856 | 9 | 5.1 | 9 | 6.3 | f | 0.808 | 1.00 | 0.80 | 0.15 |
| roh187 | rs1355913 | 10 | 5.6 | 7 | 4.9 | f | 0.808 | 0.60 | 0.70 | 0.18 |
| roh188 | rs7022766 | 4 | 2.2 | 11 | 7.6 | f | 0.031 | 1.70 | 1.10 | 0.50 |
| roh189 | rs7861200 | 14 | 7.9 | 11 | 7.6 | 0.006 | 0.940 | 1.25 | 1.25 | 0.70 |
| roh190 | rs12235141 | 7 | 3.9 | 4 | 2.8 | f | 0.760 | 1.80 | 0.70 | 0.32 |
| roh191 | rs765510 | 17 | 9.6 | 16 | 11.1 | 0.211 | 0.646 | 0.50 | 1.40 | 0.28 |
| roh192 | rs17753877 | 10 | 5.6 | 4 | 2.8 | f | 0.276 | 1.20 | 2.80 | 0.55 |
| roh193 | rs10813040 | 26 | 14.6 | 19 | 13.2 | 0.132 | 0.716 | 1.50 | 1.20 | 0.21 |
| roh194 | rs16916378 | 19 | 10.7 | 15 | 10.4 | 0.006 | 0.940 | 0.00 | 1.00 | 0.98 |
| roh195 | rs12346810 | 11 | 6.2 | 7 | 4.9 | f | 0.636 | 2.25 | 0.25 | 0.00 |
| roh196 | rs3847322 | 6 | 3.4 | 9 | 6.3 | f | 0.289 | 0.70 | 0.25 | 0.20 |
| roh197 | rs11143927 | 7 | 3.9 | 6 | 4.2 | f | 1.000 | 0.70 | 1.80 | 0.48 |
| roh198 | rs2011075 | 17 | 9.6 | 8 | 5.6 | 1.774 | 0.183 | 1.50 | 0.60 | 0.28 |
| roh199 | rs419574 | 23 | 12.9 | 7 | 4.9 | 6.121 | 0.013 | 1.80 | 1.25 | 0.50 |
| roh200 | rs4336667 | 12 | 6.7 | 7 | 4.9 | f | 0.635 | 0.80 | 1.75 | 0.10 |
| roh201 | rs603315 | 15 | 8.4 | 16 | 11.1 | 0.659 | 0.417 | 0.40 | 1.20 | 0.40 |
| roh202 | rs7031853 | 12 | 6.7 | 7 | 4.9 | f | 0.635 | 2.70 | 1.80 | 0.50 |
| roh203 | rs9783219 | 9 | 5.1 | 5 | 3.5 | f | 0.589 | 0.40 | 2.00 | 0.65 |
| roh204 | rs11013273 | 19 | 10.7 | 16 | 11.1 | 0.016 | 0.900 | 1.00 | 4.00 | 0.75 |
| roh205 | rs10828575 | 17 | 9.6 | 27 | 18.8 | 5.71 | 0.017 | 1.10 | 1.50 | 0.50 |
| roh206 | rs7068899 | 9 | 5.1 | 10 | 6.9 | f | 0.487 | 0.00 | 0.50 | 0.40 |
| roh207 | rs748375 | 12 | 6.7 | 6 | 4.2 | f | 0.343 | 1.75 | 0.75 | 0.55 |
| roh208 | rs2754428 | 18 | 10.1 | 12 | 8.3 | 0.298 | 0.585 | 2.10 | 1.70 | 0.00 |
| roh209 | rs754618 | 30 | 16.9 | 16 | 11.1 | 2.144 | 0.143 | 0.50 | 1.40 | 0.25 |
| roh210 | rs17176921 | 8 | 4.5 | 8 | 5.6 | f | 0.798 | 0.40 | 0.80 | 0.22 |
| roh211 | rs3011759 | 9 | 5.1 | 5.0 | 3.5 | f | 0.589 | 0.25 | 0.70 | 0.20 |
| roh212 | rs1876328 | 10 | 5.6 | 5 | 3.5 | f | 0.433 | 0.80 | 1.40 | 0.18 |
| roh213 | rs7095049 | 48 | 27.0 | 26 | 18.1 | 3.571 | 0.059 | 2.00 | 1.75 | 0.48 |
| roh214 | rs1199094 | 9 | 5.1 | 12 | 8.3 | f | 0.262 | 1.60 | 1.25 | 0.32 |
| roh215 | rs11006640 | 12 | 6.7 | 10 | 6.9 | f | 1.000 | 0.60 | 1.00 | 0.10 |
| roh216 | rs2170005 | 11 | 6.2 | 4 | 2.8 | f | 0.188 | 0.25 | 0.60 | 0.09 |
| roh217 | rs10995933 | 23 | 12.9 | 15 | 10.4 | 0.48 | 0.489 | 1.50 | 3.40 | 0.35 |
| roh218 | rs10762210 | 20 | 11.2 | 9 | 6.3 | 2.415 | 0.120 | 1.70 | 2.10 | 0.68 |
| roh219 | rs2675671 | 31 | 17.4 | 18 | 12.5 | 1.491 | 0.222 | 3.00 | 2.70 | 0.35 |
| roh220 | rs10762739 | 22 | 12.4 | 13 | 9.0 | 0.912 | 0.340 | 0.60 | 0.90 | 0.15 |
| roh221 | rs1041626 | 34 | 19.1 | 24 | 16.7 | 0.319 | 0.572 | 1.90 | 0.90 | 0.35 |
| roh222 | rs11201640 | 28 | 15.7 | 13 | 9.0 | 3.218 | 0.073 | 0.60 | 1.20 | 0.00 |
| roh223 | rs4529840 | 32 | 18.0 | 18 | 12.5 | 1.821 | 0.177 | 2.70 | 0.70 | 0.50 |
| roh224 | rs2105010 | 16 | 9.0 | 13 | 9.0 | 0 | 0.990 | 0.60 | 0.80 | 0.41 |
| roh225 | rs2683658 | 35 | 19.7 | 33 | 22.9 | 0.506 | 0.477 | 1.10 | 2.00 | 0.60 |
| roh226 | rs11193085 | 12 | 6.7 | 2 | 1.4 | f | 0.025 | 0.70 | 1.25 | 0.26 |
| roh227 | rs1441274 | 9 | 5.1 | 9 | 6.3 | f | 0.808 | 1.00 | 0.50 | 0.20 |
| roh228 | rs11194172 | 10 | 5.6 | 11 | 7.6 | f | 0.502 | 0.70 | 0.70 | 1.40 |
| roh229 | rs11194677 | 9 | 5.1 | 9 | 6.3 | f | 0.808 | 0.70 | 1.25 | 0.40 |
| roh230 | rs7096937 | 16 | 9.0 | 22 | 15.3 | 3.025 | 0.082 | 1.80 | 1.50 | 0.38 |
| roh231 | rs10490989 | 13 | 7.3 | 10 | 6.9 | 0.015 | 0.901 | 1.80 | 2.40 | 0.52 |
| roh232 | rs11199240 | 6 | 3.4 | 12 | 8.3 | f | 0.085 | 0.40 | 1.20 | 0.32 |
| roh233 | rs10788165 | 8 | 4.5 | 5 | 3.5 | f | 0.779 | 0.00 | 0.75 | 0.25 |
| roh234 | rs10767461 | 14 | 7.9 | 8 | 5.6 | f | 0.508 | 0.80 | 0.80 | 0.38 |
| roh235 | rs1491799 | 9 | 5.1 | 6 | 4.2 | f | 0.795 | 0.40 | 1.10 | 0.45 |
| roh236 | rs2207073 | 17 | 9.6 | 9 | 6.3 | 1.168 | 0.280 | 0.25 | 0.40 | 0.10 |
| roh237 | rs587876 | 17 | 9.6 | 11 | 7.6 | 0.366 | 0.545 | 0.80 | 0.50 | 0.38 |
| roh238 | rs10501219 | 59 | 33.1 | 39 | 27.1 | 1.382 | 0.240 | 1.10 | 5.10 | 0.45 |
| roh239 | rs1508523 | 12 | 6.7 | 11 | 7.6 | 0.097 | 0.756 | 1.50 | 0.80 | 0.52 |
| roh240 | rs16938306 | 13 | 7.3 | 4 | 2.8 | f | 0.083 | 0.80 | 1.25 | 0.20 |
| roh241 | rs10501329 | 61 | 34.3 | 48 | 33.3 | 0.031 | 0.860 | 2.30 | 1.75 | 0.70 |
| roh242 | rs1613887 | 26 | 14.6 | 24 | 16.7 | 0.258 | 0.612 | 1.20 | 0.70 | 0.00 |
| roh243 | rs7940789 | 25 | 14.0 | 20 | 13.9 | 0.002 | 0.968 | 0.75 | 1.70 | 0.15 |
| roh244 | rs1286289 | 21 | 11.8 | 16 | 11.1 | 0.037 | 0.848 | 0.50 | 2.00 | 0.00 |
| roh245 | rs633568 | 9 | 5.1 | 7 | 4.9 | f | 1.000 | 0.75 | 1.10 | 0.30 |
| roh246 | rs10830207 | 20 | 11.2 | 12 | 8.3 | 0.749 | 0.387 | 1.00 | 0.75 | 0.30 |
| roh247 | rs10830716 | 17 | 9.6 | 15 | 10.4 | 0.067 | 0.796 | 1.30 | 1.30 | 0.38 |
| roh248 | rs7945975 | 7 | 3.9 | 13 | 9.0 | f | 0.067 | 0.00 | 0.40 | 0.10 |
| roh249 | rs1792622 | 10 | 5.6 | 9 | 6.3 | f | 0.817 | 0.25 | 2.25 | 0.36 |
| roh250 | rs11225877 | 14 | 7.9 | 0 | 0.0 | f | 0.000 | 0.25 | 1.30 | 0.25 |
| roh251 | rs638266 | 38 | 21.3 | 23 | 16.0 | 1.498 | 0.221 | 1.75 | 1.25 | 0.72 |
| roh252 | rs17115275 | 12 | 6.7 | 5 | 3.5 | f | 0.219 | 0.25 | 0.70 | 0.12 |
| roh253 | rs956959 | 26 | 14.6 | 14 | 9.7 | 1.746 | 0.186 | 0.25 | 1.25 | 0.35 |
| roh254 | rs7297320 | 7 | 3.9 | 7 | 4.9 | f | 0.786 | 1.90 | 1.80 | 0.58 |
| roh255 | rs11049731 | 4 | 2.2 | 10 | 6.9 | f | 0.053 | 0.00 | 0.30 | 0.08 |
| roh256 | rs16906504 | 6 | 3.4 | 10 | 6.9 | f | 0.197 | 0.80 | 0.20 | 0.25 |
| roh257 | rs11053146 | 7 | 3.9 | 10 | 6.9 | f | 0.316 | 1.10 | 0.60 | 0.58 |
| roh258 | rs7486293 | 18 | 10.1 | 14 | 9.7 | 0.014 | 0.907 | 1.90 | 2.00 | 0.25 |
| roh259 | rs1908592 | 14 | 7.9 | 12 | 8.3 | 0.024 | 0.878 | 1.40 | 1.10 | 0.18 |
| roh260 | rs17094772 | 7 | 3.9 | 11 | 7.6 | f | 0.222 | 0.60 | 1.75 | 0.32 |
| roh261 | rs10161093 | 8 | 4.5 | 5 | 3.5 | f | 0.779 | 0.40 | 1.20 | 0.62 |
| roh262 | rs10875842 | 5 | 2.8 | 7 | 4.9 | f | 0.384 | 1.80 | 2.00 | 0.22 |
| roh263 | rs711330 | 11 | 6.2 | 7 | 4.9 | f | 0.636 | 0.25 | 0.70 | 0.25 |
| roh264 | rs17122290 | 9 | 5.1 | 9 | 6.3 | f | 0.808 | 0.25 | 1.10 | 0.58 |
| roh265 | rs11173469 | 9 | 5.1 | 6 | 4.2 | f | 0.795 | 0.70 | 0.70 | 0.18 |
| roh266 | rs6581758 | 8 | 4.5 | 4 | 2.8 | f | 0.558 | 0.25 | 0.60 | 2.40 |
| roh267 | rs11180138 | 11 | 6.2 | 11 | 7.6 | f | 0.660 | 1.80 | 0.70 | 0.68 |
| roh268 | rs4143624 | 16 | 9.0 | 10 | 6.9 | 0.448 | 0.503 | 2.24 | 1.40 | 4.40 |
| roh269 | rs10735441 | 14 | 7.9 | 9 | 6.3 | 0.313 | 0.576 | 1.20 | 2.50 | 0.35 |
| roh270 | rs7977839 | 20 | 11.2 | 21 | 14.6 | 0.803 | 0.370 | 0.60 | 0.70 | 0.80 |
| roh271 | rs17014369 | 16 | 9.0 | 9 | 6.3 | 0.834 | 0.361 | 1.25 | 2.60 | 0.68 |
| roh272 | rs17016387 | 42 | 23.6 | 35 | 24.3 | 0.022 | 0.882 | 0.70 | 1.50 | 0.50 |
| roh273 | rs11106228 | 14 | 7.9 | 10 | 6.9 | 0.098 | 0.754 | 2.00 | 0.70 | 0.08 |
| roh274 | rs17026179 | 14 | 7.9 | 6 | 4.2 | f | 0.245 | 1.20 | 0.80 | 0.00 |
| roh275 | rs10850084 | 42 | 23.6 | 37 | 25.7 | 0.189 | 0.663 | 1.10 | 1.80 | 0.22 |
| roh276 | rs11066960 | 11 | 6.2 | 6 | 4.2 | f | 0.464 | 0.40 | 0.40 | 0.18 |
| roh277 | rs4415922 | 14 | 7.9 | 12 | 8.3 | 0.024 | 0.878 | 1.20 | 1.75 | 0.52 |
| roh278 | rs9283079 | 10 | 5.6 | 7 | 4.9 | f | 0.808 | 0.90 | 1.25 | 0.09 |
| roh279 | rs9537441 | 23 | 12.9 | 14 | 9.7 | 0.801 | 0.371 | 0.75 | 0.75 | 0.45 |
| roh280 | rs7992804 | 16 | 9.0 | 7 | 4.9 | 2.045 | 0.153 | 1.50 | 2.50 | 0.25 |
| roh281 | rs12431035 | 7 | 3.9 | 9 | 6.3 | f | 0.441 | 1.20 | 1.00 | 0.00 |
| roh282 | rs1770325 | 10 | 5.6 | 8 | 5.6 | f | 1.000 | 1.10 | 1.40 | 0.22 |
| roh283 | rs9560362 | 14 | 7.9 | 7 | 4.9 | f | 0.365 | 0.90 | 0.75 | 0.00 |
| roh284 | rs9516766 | 15 | 8.4 | 5 | 3.5 | f | 0.102 | 0.50 | 1.50 | 0.45 |
| roh285 | rs1957583 | 8 | 4.5 | 7 | 4.9 | f | 1.000 | 0.25 | 1.50 | 0.50 |
| roh286 | rs10150617 | 4 | 2.2 | 11 | 7.6 | f | 0.031 | 0.25 | 0.40 | 0.40 |
| roh287 | rs12147034 | 21 | 11.8 | 16 | 11.1 | 0.037 | 0.848 | 0.60 | 2.10 | 0.38 |
| roh288 | rs11158366 | 14 | 7.9 | 15 | 10.4 | 0.632 | 0.426 | 2.40 | 2.00 | 0.80 |
| roh289 | rs7144688 | 5 | 2.8 | 9 | 6.3 | f | 0.171 | 1.60 | 2.10 | 0.68 |
| roh290 | rs17825846 | 13 | 7.3 | 5 | 3.5 | f | 0.152 | 1.10 | 1.20 | 0.42 |
| roh291 | rs12437164 | 86 | 48.3 | 47 | 32.6 | 8.068 | 0.005 | 2.50 | 2.75 | 0.99 |
| roh292 | rs7359118 | 23 | 12.9 | 15 | 10.4 | 0.48 | 0.489 | 0.40 | 1.10 | 0.30 |
| roh293 | rs1892225 | 7 | 3.9 | 11 | 7.6 | f | 0.222 | 0.90 | 1.10 | 0.18 |
| roh294 | rs3784391 | 8 | 4.5 | 6 | 4.2 | f | 1.000 | 1.10 | 1.30 | 0.65 |
| roh295 | rs11637483 | 31 | 17.4 | 17 | 11.8 | 1.975 | 0.160 | 1.60 | 3.20 | 0.90 |
| roh296 | rs4381535 | 42 | 23.6 | 23 | 16.0 | 2.871 | 0.090 | 1.80 | 2.50 | 0.70 |
| roh297 | rs2445765 | 6 | 3.4 | 7 | 4.9 | f | 0.575 | 0.70 | 0.30 | 0.18 |
| roh298 | rs8034032 | 13 | 7.3 | 9 | 6.3 | f | 0.825 | 2.10 | 1.75 | 0.38 |
| roh299 | rs1073097 | 17 | 9.6 | 16 | 11.1 | 0.211 | 0.646 | 0.70 | 0.60 | 0.10 |
| roh300 | rs4777180 | 18 | 10.1 | 12 | 8.3 | 0.298 | 0.585 | 1.60 | 2.60 | 1.25 |
| roh301 | rs16957968 | 29 | 16.3 | 19 | 13.2 | 0.602 | 0.438 | 2.20 | 2.80 | 0.78 |
| roh302 | rs17382798 | 7 | 3.9 | 7 | 4.9 | f | 0.786 | 1.50 | 1.40 | 0.21 |
| roh303 | rs11635431 | 11 | 6.2 | 5 | 3.5 | f | 0.311 | 0.40 | 0.80 | 0.15 |
| roh304 | rs12595866 | 7 | 3.9 | 6 | 4.2 | f | 1.000 | 0.40 | 0.60 | 0.21 |
| roh305 | rs10500375 | 11 | 6.2 | 9 | 6.3 | f | 1.000 | 0.40 | 0.60 | 0.00 |
| roh306 | rs8046716 | 38 | 21.3 | 21 | 14.6 | 2.434 | 0.119 | 0.90 | 4.25 | 0.35 |
| roh307 | rs13329873 | 9 | 5.1 | 8 | 5.6 | f | 1.000 | 1.50 | 0.30 | 0.10 |
| roh308 | rs4783573 | 29 | 16.3 | 23 | 16.0 | 0.006 | 0.938 | 1.10 | 2.60 | 0.80 |
| roh309 | ? | 7 | 3.9 | 6 | 4.2 | f | 1.000 | 1.30 | 1.10 | 0.10 |
| roh310 | ? | 23 | 12.9 | 17 | 11.8 | 0.091 | 0.763 | 2.25 | 2.20 | 0.50 |
| roh311 | rs1518603 | 24 | 13.5 | 15 | 10.4 | 0.703 | 0.402 | 1.80 | 1.20 | 0.60 |
| roh312 | rs7205794 | 11 | 6.2 | 5 | 3.5 | f | 0.311 | 1.00 | 1.00 | 0.45 |
| roh313 | rs4796675 | 18 | 10.1 | 17 | 11.8 | 0.236 | 0.627 | 0.80 | 1.10 | 0.40 |
| roh314 | rs11570441 | 40 | 22.5 | 14 | 9.7 | 9.271 | 0.002 | 2.50 | 0.75 | 0.35 |
| roh315 | rs9898730 | 14 | 7.9 | 15 | 10.4 | 0.632 | 0.426 | 1.70 | 0.70 | 0.28 |
| roh316 | rs9912513 | 23 | 12.9 | 18 | 12.5 | 0.013 | 0.910 | 0.90 | 1.70 | 0.70 |
| roh317 | rs12948356 | 24 | 13.5 | 13 | 9.0 | 1.554 | 0.213 | 0.60 | 2.60 | 0.72 |
| roh318 | rs7235201 | 9 | 5.1 | 4 | 2.8 | f | 0.398 | 0.80 | 1.10 | 0.55 |
| roh319 | rs16946546 | 13 | 7.3 | 8 | 5.6 | f | 0.652 | 1.60 | 0.75 | 0.00 |
| roh320 | rs1562995 | 18 | 10.1 | 12 | 8.3 | 0.298 | 0.585 | 0.40 | 1.50 | 0.32 |
| roh321 | rs16975303 | 15 | 8.4 | 1 | 0.7 | f | 0.001 | 2.25 | 1.60 | 0.24 |
| roh322 | rs610325 | 21 | 11.8 | 10 | 6.9 | 2.155 | 0.142 | 0.60 | 0.80 | 0.10 |
| roh323 | rs11083005 | 16 | 9.0 | 16 | 11.1 | 0.401 | 0.527 | 1.25 | 0.70 | 0.38 |
| roh324 | rs1420766 | 17 | 9.6 | 16 | 11.1 | 0.211 | 0.646 | 0.70 | 0.40 | 0.10 |
| roh325 | rs17069904 | 13 | 7.3 | 3 | 2.1 | f | 0.039 | 0.00 | 1.40 | 0.25 |
| roh326 | rs11085468 | 7 | 3.9 | 8 | 5.6 | f | 0.598 | 0.80 | 1.40 | 0.38 |
| roh327 | rs7252575 | 28 | 15.7 | 17 | 11.8 | 1.02 | 0.313 | 1.20 | 2.00 | 0.25 |
| roh328 | rs10412480 | 11 | 6.2 | 10 | 6.9 | f | 0.823 | 0.60 | 1.80 | 0.08 |
| roh329 | rs833915 | 7 | 3.9 | 5 | 3.5 | f | 1.000 | 0.80 | 0.80 | 0.42 |
| roh330 | rs742853 | 25 | 14.0 | 32 | 22.2 | 3.654 | 0.056 | 2.50 | 1.75 | 0.40 |
| roh331 | rs6049605 | 9 | 5.1 | 6 | 4.2 | f | 0.795 | 1.10 | 1.60 | 0.42 |
| roh332 | rs6015104 | 20 | 11.2 | 22 | 15.3 | 1.147 | 0.284 | 2.50 | 1.80 | 0.38 |
| roh333 | rs6071828 | 28 | 15.7 | 14 | 9.7 | 2.533 | 0.111 | 1.25 | 1.20 | 0.45 |
| roh334 | ? | 22 | 12.4 | 16 | 11.1 | 0.119 | 0.730 | 1.00 | 0.70 | 0.10 |
| roh335 | rs4811599 | 17 | 9.6 | 6 | 4.2 | 3.479 | 0.062 | 1.75 | 1.75 | 0.55 |
| roh336 | rs2829931 | 16 | 9.0 | 6 | 4.2 | f | 0.119 | 1.10 | 1.50 | 0.20 |
| roh337 | rs8129810 | 24 | 13.5 | 22 | 15.3 | 0.209 | 0.647 | 2.24 | 2.25 | 0.58 |
| roh338 | rs12484740 | 14 | 7.9 | 15 | 10.4 | 0.632 | 0.426 | 1.60 | 2.00 | 0.30 |
| roh339 | rs5998365 | 13 | 7.3 | 10 | 6.9 | 0.015 | 0.901 | 1.20 | 1.10 | 0.42 |
| roh340 | rs17002946 | 16 | 9.0 | 9 | 6.3 | 0.834 | 0.361 | 0.90 | 1.30 | 0.30 |

SUPPLEMENTARY TABLE 2

| Affy_ID | dbSNP | Chr | band | Position | Gene | P-value |
|---|---|---|---|---|---|---|
| SNP_A-2056719 | rs2923062 | 8 | q11.21 | 51441469 | SNTG1 | 6.69E−03 |
| SNP_A-2303509 | rs16914780 | 8 | q11.21 | 51449609 | SNTG1 | 6.69E−03 |
| SNP_A-2193025 | rs16914781 | 8 | q11.21 | 51450035 | SNTG1 | 6.69E−03 |
| SNP_A-4204516 | rs4534138 | 8 | q11.21 | 51451367 | SNTG1 | 6.69E−03 |
| SNP_A-2281233 | rs10957894 | 8 | q11.21 | 51454425 | SNTG1 | 7.99E−03 |
| SNP_A-2057710 | rs4292700 | 8 | q11.21 | 51455302 | SNTG1 | 7.99E−03 |
| SNP_A-1956376 | rs7823310 | 8 | q11.21 | 51456091 | SNTG1 | 7.99E−03 |
| SNP_A-2095646 | rs6473111 | 8 | q11.21 | 51458884 | SNTG1 | 7.99E−03 |
| SNP_A-2198909 | rs7839253 | 8 | q11.21 | 51459986 | SNTG1 | 7.99E−03 |
| SNP_A-4282968 | rs4524809 | 8 | q11.21 | 51462851 | SNTG1 | 7.99E−03 |
| SNP_A-1917516 | rs4339654 | 8 | q11.21 | 51463114 | SNTG1 | 7.99E−03 |
| SNP_A-1858682 | rs4529480 | 8 | q11.21 | 51469016 | SNTG1 | 7.99E−03 |
| SNP_A-1961465 | rs4563896 | 8 | q11.21 | 51473631 | SNTG1 | 3.97E−03 |
| SNP_A-2266190 | rs7820992 | 8 | q11.21 | 51473649 | SNTG1 | 3.97E−03 |
| SNP_A-2206873 | rs4242461 | 8 | q11.21 | 51475032 | SNTG1 | 1.78E−03 |
| SNP_A-2146318 | rs7843301 | 8 | q11.21 | 51498389 | SNTG1 | 1.78E−03 |
| SNP_A-4266939 | rs2062039 | 8 | q11.21 | 51502012 | SNTG1 | 1.78E−03 |
| SNP_A-2150690 | rs1481467 | 8 | q11.21 | 51506089 | SNTG1 | 2.67E−03 |
| SNP_A-4203395 | rs9650165 | 8 | q11.21 | 51506205 | SNTG1 | 2.53E−03 |
| SNP_A-2150643 | rs10957915 | 8 | q11.21 | 51511311 | SNTG1 | 2.53E−03 |
| SNP_A-2132611 | rs10957916 | 8 | q11.21 | 51511374 | SNTG1 | 2.53E−03 |
| SNP_A-2075720 | rs1471578 | 8 | q11.21 | 51523063 | SNTG1 | 1.70E−03 |
| SNP_A-1895133 | rs9987391 | 8 | q11.21 | 51523500 | SNTG1 | 1.70E−03 |
| SNP_A-4276525 | rs4873147 | 8 | q11.21 | 51524267 | SNTG1 | 1.70E−03 |
| SNP_A-4277171 | rs4873458 | 8 | q11.21 | 51524300 | SNTG1 | 1.70E−03 |
| SNP_A-4228253 | rs10088756 | 8 | q11.21 | 51528317 | SNTG1 | 1.70E−03 |
| SNP_A-1961356 | rs1383819 | 8 | q11.21 | 51534019 | SNTG1 | 3.73E−03 |
| SNP_A-4217136 | rs1904997 | 8 | q11.21 | 51541878 | SNTG1 | 3.73E−03 |
| SNP_A-4237065 | rs4440649 | 8 | q11.21 | 51542530 | SNTG1 | 1.78E−03 |
| SNP_A-2022493 | rs12547263 | 8 | q11.21 | 51552222 | SNTG1 | 1.78E−03 |
| SNP_A-1801379 | rs1481472 | 8 | q11.21 | 51557371 | SNTG1 | 1.78E−03 |
| SNP_A-1993218 | rs2392699 | 8 | q11.21 | 51563239 | SNTG1 | 1.78E−03 |
| SNP_A-2265987 | rs2467203 | 8 | q11.21 | 51605982 | SNTG1 | 1.21E−03 |
| SNP_A-2184475 | ? | 8 | q11.21 | 51610161 | SNTG1 | 1.21E−03 |
| SNP_A-1874720 | rs1542615 | 8 | q11.21 | 51616502 | SNTG1 | 1.21E−03 |
| SNp_A-1921640 | rs2623207 | 8 | q11.21 | 51620897 | SNTG1 | 1.21E−03 |
| SNP_A-2046087 | rs2623225 | 8 | q11.21 | 51622219 | SNTG1 | 1.21E−03 |
| SNP_A-4254534 | rs2625758 | 8 | q11.21 | 51624716 | SNTG1 | 5.10E−04 |
| SNP_A-2152914 | rs2623224 | 8 | q11.21 | 51624769 | SNTG1 | 3.28E−04 |
| SNP_A-1923956 | rs16915033 | 8 | q11.21 | 51625180 | SNTG1 | 7.57E−04 |
| SNP_A-2238756 | rs10107280 | 8 | q11.21 | 51631958 | SNTG1 | 7.57E−04 |
| SNP_A-2037049 | rs1483640 | 8 | q11.21 | 51633485 | SNTG1 | 1.81E−03 |
| SNP_A-2293183 | rs1351754 | 8 | q11.21 | 51651167 | SNTG1 | 1.81E−03 |
| SNP_A-1937391 | rs1580413 | 8 | q11.21 | 51651512 | SNTG1 | 1.81E−03 |
| SNP_A-4253129 | rs996166 | 8 | q11.21 | 51657369 | SNTG1 | 4.24E−03 |
| SNP_A-4207289 | rs1483638 | 8 | q11.21 | 51681895 | SNTG1 | 2.81E−03 |
| SNP_A-1825303 | rs6473253 | 8 | q11.21 | 51684525 | SNTG1 | 2.81E−03 |
| SNP_A-2153675 | rs6998737 | 8 | q11.21 | 51693370 | SNTG1 | 2.81E−03 |
| SNP_A-2094331 | rs16915106 | 8 | q11.21 | 51699950 | SNTG1 | 2.81E−03 |
| SNP_A-4206075 | rs11991826 | 8 | q11.21 | 51701087 | SNTG1 | 2.81E−03 |
| SNP_A-1933938 | rs16915112 | 8 | q11.21 | 51701435 | SNTG1 | 2.81E−03 |
| SNP_A-4266940 | rs1384830 | 8 | q11.21 | 51707146 | SNTG1 | 2.81E−03 |
| SNP_A-1993221 | rs10216995 | 8 | q11.21 | 51712398 | SNTG1 | 2.81E−03 |
| SNP_A-1791334 | rs4401874 | 8 | q11.21 | 51719839 | SNTG1 | 4.24E−03 |
| SNP_A-2062612 | rs1384831 | 8 | q11.21 | 51723458 | SNTG1 | 4.24E−03 |
| SNP_A-1993222 | rs10110909 | 8 | q11.21 | 51727199 | SNTG1 | 9.48E−03 |
| SNP_A-2264825 | rs6473289 | 8 | q11.21 | 51756441 | SNTG1 | 6.17E−03 |
| SNP_A-2151001 | rs1396377 | 8 | q11.21 | 51756680 | SNTG1 | 6.17E−03 |
| SNP_A-1914476 | rs6473290 | 8 | q11.21 | 51759364 | SNTG1 | 6.17E−03 |
| SNP_A-2086177 | rs7016914 | 8 | q11.21 | 51780168 | SNTG1 | 6.17E−03 |
| SNP_A-2265202 | ? | 8 | q11.21 | 51793164 | SNTG1 | 6.17E−03 |
| SNP_A-1902908 | rs1911832 | 8 | q11.21 | 51797066 | SNTG1 | 6.17E−03 |
| SNP_A-1905567 | rs6985954 | 8 | q11.21 | 51798428 | SNTG1 | 2.81E−03 |
| SNP_A-2081526 | rs6473320 | 8 | q11.21 | 51804559 | SNTG1 | 2.81E−03 |
| SNP_A-1993223 | rs906656 | 8 | q11.21 | 51805005 | SNTG1 | 2.81E−03 |
| SNP_A-4259350 | rs11986411 | 8 | q11.21 | 51816608 | SNTG1 | 2.81E−03 |
| SNP_A-2013388 | rs7831651 | 8 | q11.21 | 51859645 | SNTG1 | 4.24E−03 |
| SNP_A-1958710 | rs7832680 | 8 | q11.21 | 51859663 | SNTG1 | 4.24E−03 |
| SNP_A-1819627 | rs7832799 | 8 | q11.21 | 51859715 | SNTG1 | 4.24E−03 |
| SNP_A-2126502 | ? | 8 | q11.21 | 51866630 | SNTG1 | 4.24E−03 |
| SNP_A-4295444 | rs7008324 | 8 | q11.21 | 51883526 | intergenic | 2.81E−03 |
| SNP_A-4199849 | rs1484126 | 8 | q11.21 | 51884347 | intergenic | 2.81E−03 |
| SNP_A-1882648 | rs1484127 | 8 | q11.21 | 51888207 | intergenic | 2.81E−03 |
| SNP_A-2260759 | rs1484128 | 8 | q11.21 | 51888421 | intergenic | 2.81E−03 |
| SNP_A-1855713 | rs6473406 | 8 | q11.21 | 51900282 | intergenic | 2.81E−03 |
| SNP_A-2258910 | rs1484129 | 8 | q11.21 | 51900540 | intergenic | 2.81E−03 |
| SNP_A-4234491 | rs2392742 | 8 | q11.21 | 51932422 | intergenic | 2.81E−03 |
| SNP_A-2147522 | rs4520181 | 8 | q11.21 | 51932442 | intergenic | 1.85E−03 |
| SNP_A-2083927 | rs7013653 | 8 | q11.21 | 51933484 | intergenic | 1.85E−03 |
| SNP_A-1783958 | rs4633069 | 8 | q11.21 | 51939038 | intergenic | 1.85E−03 |
| SNP_A-2295301 | rs1601194 | 8 | q11.21 | 51941043 | intergenic | 1.85E−03 |
| SNP_A-4245626 | rs975382 | 8 | q11.21 | 51945468 | intergenic | 1.85E−03 |
| SNP_A-1791182 | rs10283050 | 8 | q11.21 | 51949988 | intergenic | 1.85E−03 |
| SNP_A-4243596 | rs10283046 | 8 | q11.21 | 51950419 | intergenic | 1.85E−03 |
| SNP_A-4271145 | rs7016107 | 8 | q11.21 | 51951872 | intergenic | 1.85E−03 |
| SNP_A-1817118 | rs10102886 | 8 | q11.21 | 51968128 | intergenic | 1.85E−03 |
| SNP_A-1868040 | rs6987928 | 8 | q11.21 | 51977243 | intergenic | 1.85E−03 |
| SNP_A-2045171 | rs11994633 | 8 | q11.21 | 51979461 | intergenic | 1.85E−03 |
| SNP_A-4206004 | rs6473486 | 8 | q11.21 | 51979598 | intergenic | 1.85E−03 |
| SNP_A-1993224 | rs10504109 | 8 | q11.21 | 51997438 | intergenic | 1.85E−03 |
| SNP_A-1880357 | rs13281139 | 8 | q11.21 | 52006606 | intergenic | 1.85E−03 |
| SNP_A-1920800 | rs1159997 | 8 | q11.21 | 52021786 | intergenic | 4.14E−03 |
| SNP_A-4290353 | rs7837741 | 8 | q11.21 | 52027657 | intergenic | 4.14E−03 |
| SNP_A-2204830 | rs1905526 | 8 | q11.21 | 52032418 | intergenic | 4.14E−03 |
| SNP_A-2291000 | rs6989442 | 8 | q11.21 | 52036135 | intergenic | 4.14E−03 |
| SNP_A-2260772 | rs9969399 | 8 | q11.21 | 52037226 | intergenic | 4.14E−03 |
| SNP_A-1883909 | rs1484130 | 8 | q11.21 | 52045960 | intergenic | 4.14E−03 |
| SNP_A-1896767 | rs2168331 | 8 | q11.21 | 52070083 | intergenic | 4.14E−03 |
| SNP_A-4202203 | rs2219209 | 8 | q11.21 | 52079813 | intergenic | 4.14E−03 |
| SNP_A-1937329 | rs2392765 | 8 | q11.21 | 52079874 | intergenic | 4.14E−03 |
| SNP_A-1920731 | rs6473561 | 8 | q11.21 | 52080057 | intergenic | 9.11E−03 |
| SNP_A-2064652 | rs16915710 | 8 | q11.21 | 52091409 | intergenic | 6.17E−03 |
| SNP_A-4196531 | rs7009881 | 8 | q11.21 | 52092215 | intergenic | 6.17E−03 |
| SNP_A-2024615 | rs10958238 | 8 | q11.21 | 52109252 | intergenic | 6.17E−03 |
| SNP_A-1826896 | rs10106393 | 8 | q11.21 | 52120991 | intergenic | 6.17E−03 |
| SNP_A-2060292 | rs6473574 | 8 | q11.21 | 52122112 | intergenic | 6.17E−03 |
| SNP_A-4222443 | rs17728090 | 8 | q11.21 | 52132300 | intergenic | 6.17E−03 |
| SNP_A-1938600 | rs6993652 | 8 | q11.21 | 52154155 | intergenic | 6.17E−03 |
| SNP_A-1792544 | rs11192943 | 10 | q25.1 | 108287891 | intergenic | 6.12E−03 |
| SNP_A-2021825 | rs7088535 | 10 | q25.1 | 108288081 | intergenic | 6.12E−03 |
| SNP_A-1782767 | rs17297851 | 10 | q25.1 | 108289414 | intergenic | 6.12E−03 |
| SNP_A-2311304 | rs17297858 | 10 | q25.1 | 108289455 | intergenic | 6.12E−03 |
| SNP_A-2028442 | rs11192958 | 10 | q25.1 | 108317321 | intergenic | 6.12E−03 |
| SNP_A-2053519 | rs12359404 | 10 | q25.1 | 108324179 | SORCS1 | 6.12E−03 |
| SNP_A-2004985 | rs10491050 | 10 | q25.1 | 108325998 | SORCS1 | 6.12E−03 |
| SNP_A-2048295 | rs11192967 | 10 | q25.1 | 108329322 | SORCS1 | 6.12E−03 |
| SNP_A-1830517 | rs11192968 | 10 | q25.1 | 108330433 | SORCS1 | 1.50E−03 |
| SNP_A-1940703 | rs4917477 | 10 | q25.1 | 108336217 | SORCS1 | 2.40E−03 |
| SNP_A-1819847 | rs17120993 | 10 | q25.1 | 108337180 | SORCS1 | 2.40E−03 |
| SNP_A-2251198 | rs17121023 | 10 | q25.1 | 108342175 | SORCS1 | 2.40E−03 |
| SNP_A-1956347 | rs11814145 | 10 | q25.1 | 108345833 | SORCS1 | 2.40E−03 |
| SNP_A-2048623 | rs1269918 | 10 | q25.1 | 108345864 | SORCS1 | 2.40E−03 |
| SNP_A-2050923 | rs11817694 | 10 | q25.1 | 108348488 | SORCS1 | 2.40E−03 |
| SNP_A-1951360 | rs11192973 | 10 | q25.1 | 108353452 | SORCS1 | 2.40E−03 |
| SNP_A-4214438 | rs11192974 | 10 | q25.1 | 108354288 | SORCS1 | 2.40E−03 |
| SNP_A-4214439 | rs11817663 | 10 | q25.1 | 108354299 | SORCS1 | 2.40E−03 |
| SNP_A-2004989 | rs10884335 | 10 | q25.1 | 108354793 | SORCS1 | 2.40E−03 |
| SNP_A-1888019 | rs6584758 | 10 | q25.1 | 108359502 | SORCS1 | 2.40E−03 |
| SNP_A-2153850 | rs10884337 | 10 | q25.1 | 108366461 | SORCS1 | 2.40E−03 |
| SNP_A-1922461 | rs1322006 | 10 | q25.1 | 108370516 | SORCS1 | 2.40E−03 |
| SNP_A-2246290 | rs1322005 | 10 | q25.1 | 108370669 | SORCS1 | 2.40E−03 |
| SNP_A-4222780 | rs7074484 | 10 | q25.1 | 108371247 | SORCS1 | 2.40E−03 |
| SNP_A-2035855 | rs7914387 | 10 | q25.1 | 108371398 | SORCS1 | 2.40E−03 |
| SNP_A-2104084 | rs7901090 | 10 | q25.1 | 108382032 | SORCS1 | 2.40E−03 |
| SNP_A-2291700 | rs11192994 | 10 | q25.1 | 108398096 | SORCS1 | 2.40E−03 |
| SNP_A-2312317 | rs11192997 | 10 | q25.1 | 108400332 | SORCS1 | 2.40E−03 |
| SNP_A-1815743 | rs11192998 | 10 | q25.1 | 108400575 | SORCS1 | 2.40E−03 |
| SNP_A-2050336 | rs11193000 | 10 | q25.1 | 108404270 | SORCS1 | 2.40E−03 |
| SNP_A-1851132 | rs12254438 | 10 | q25.1 | 108409046 | SORCS1 | 2.40E−03 |
| SNP_A-2196714 | rs7095966 | 10 | q25.1 | 108410576 | SORCS1 | 2.40E−03 |
| SNP_A-2041150 | rs11193005 | 10 | q25.1 | 108411130 | SORCS1 | 2.40E−03 |
| SNP_A-1852117 | rs11598223 | 10 | q25.1 | 108411946 | SORCS1 | 2.40E−03 |
| SNP_A-1869058 | rs9633679 | 10 | q25.1 | 108412110 | SORCS1 | 2.40E−03 |
| SNP_A-2258251 | rs11193007 | 10 | q25.1 | 108414751 | SORCS1 | 2.40E−03 |
| SNP_A-2001412 | rs821931 | 10 | q25.1 | 108427629 | SORCS1 | 2.40E−03 |
| SNP_A-2233066 | rs821925 | 10 | q25.1 | 108432009 | SORCS1 | 2.40E−03 |
| SNP_A-2157313 | rs821943 | 10 | q25.1 | 108432517 | SORCS1 | 2.40E−03 |
| SNP_A-2115676 | rs12256633 | 10 | q25.1 | 108433366 | SORCS1 | 2.40E−03 |
| SNP_A-4250999 | rs821940 | 10 | q25.1 | 108434959 | SORCS1 | 2.40E−03 |
| SNP_A-4272241 | rs821934 | 10 | q25.1 | 108438994 | SORCS1 | 2.40E−03 |
| SNP_A-1921494 | rs821958 | 10 | q25.1 | 108445133 | SORCS1 | 2.40E−03 |
| SNP_A-2236831 | rs12243064 | 10 | q25.1 | 108464605 | SORCS1 | 2.40E−03 |
| SNP_A-1785958 | rs12250100 | 10 | q25.1 | 108464855 | SORCS1 | 2.40E−03 |
| SNP_A-2086159 | rs12267167 | 10 | q25.1 | 108466097 | SORCS1 | 2.40E−03 |
| SNP_A-1898273 | rs10884341 | 10 | q25.1 | 108469639 | SORCS1 | 2.40E−03 |
| SNP_A-2255102 | rs11193022 | 10 | q25.1 | 108470389 | SORCS1 | 2.40E−03 |
| SNP_A-4279933 | rs1040871 | 10 | q25.1 | 108474359 | SORCS1 | 2.40E−03 |
| SNP_A-2069915 | rs1358874 | 10 | q25.1 | 108474505 | SORCS1 | 2.40E−03 |
| SNP_A-1896037 | rs10786967 | 10 | q25.1 | 108483941 | SORCS1 | 2.40E−03 |
| SNP_A-1886734 | rs10884345 | 10 | q25.1 | 108484098 | SORCS1 | 2.40E−03 |
| SNP_A-2132861 | rs7903481 | 10 | q25.1 | 108489892 | SORCS1 | 2.40E−03 |
| SNP_A-4245944 | rs10884350 | 10 | q25.1 | 108490065 | SORCS1 | 2.40E−03 |
| SNP_A-4239956 | rs10458732 | 10 | q25.1 | 108490394 | SORCS1 | 2.40E−03 |
| SNP_A-2004997 | rs9325521 | 10 | q25.1 | 108490722 | SORCS1 | 2.40E−03 |
| SNP_A-2004999 | rs1890457 | 10 | q25.1 | 108511556 | SORCS1 | 2.40E−03 |
| SNP_A-2005001 | rs999776 | 10 | q25.1 | 108516800 | SORCS1 | 2.40E−03 |
| SNP_A-2115027 | rs12771665 | 10 | q25.1 | 108521546 | SORCS1 | 2.40E−03 |
| SNP_A-2218082 | rs2484972 | 10 | q25.1 | 108521598 | SORCS1 | 2.40E−03 |
| SNP_A-1785362 | rs11193042 | 10 | q25.1 | 108521694 | SORCS1 | 2.40E−03 |
| SNP_A-4247707 | rs945598 | 10 | q25.1 | 108522614 | SORCS1 | 2.40E−03 |
| SNP_A-4254338 | rs12779808 | 10 | q25.1 | 108522767 | SORCS1 | 2.40E−03 |
| SNP_A-4282066 | rs878182 | 10 | q25.1 | 108523116 | SORCS1 | 2.40E−03 |
| SNP_A-2005003 | rs10509821 | 10 | q25.1 | 108524996 | SORCS1 | 2.40E−03 |
| SNP_A-4268792 | rs10509822 | 10 | q25.1 | 108525418 | SORCS1 | 2.40E−03 |
| SNP_A-1802885 | rs2245123 | 10 | q25.1 | 108543204 | SORCS1 | 2.40E−03 |
| SNP_A-1820891 | rs2255917 | 10 | q25.1 | 108546112 | SORCS1 | 2.40E−03 |
| SNP_A-1858009 | rs2486154 | 10 | q25.1 | 108549689 | SORCS1 | 2.40E−03 |
| SNP_A-2283854 | rs4917487 | 10 | q25.1 | 108550789 | SORCS1 | 2.40E−03 |
| SNP_A-2154229 | rs12248379 | 10 | q25.1 | 108551998 | SORCS1 | 2.40E−03 |
| SNP_A-4201161 | rs11193059 | 10 | q25.1 | 108562383 | SORCS1 | 2.40E−03 |
| SNP_A-1843438 | rs1538417 | 10 | q25.1 | 108573589 | SORCS1 | 2.40E−03 |
| SNP_A-4202792 | rs11597875 | 10 | q25.1 | 108580272 | SORCS1 | 2.40E−03 |
| SNP_A-2084521 | rs7087219 | 10 | q25.1 | 108611655 | SORCS1 | 2.40E−03 |
| SNP_A-4216596 | rs874887 | 10 | q25.1 | 108613957 | SORCS1 | 2.40E−03 |
| SNP_A-2188906 | rs1336615 | 10 | q25.1 | 108616599 | SORCS1 | 3.86E−03 |
| SNP_A-2081598 | rs11193085 | 10 | q25.1 | 108623596 | SORCS1 | 3.86E−03 |

### REFERENCES

Allen N C, Bagade S, Tanzi R, Bertram L (Accessed May 2, 2007) The SchizophreniaGene Database. Schizophrenia Research Forum. Available at: http://www.schizophreniaforum.org/res/sczgene/default.asp
Brzustowicz L M, Simone J, Mohseni P, Hayter J E, Hodgkinson K A, Chow E W, Bassett A S (2004) Linkage disequilibrium mapping of schizophrenia susceptibility to the CAPON region of chromosome 1q22. Am J Hum Genet. 74:1057-63
Carlson C S, Eberle M A, Kruglyak L, Nickerson D A (2004) Mapping complex disease loci in whole-genome association studies. Nature 429:446-452
Clark A G, Hubisz M J, Bustamante C D, Williamson S H, Nielsen R (2005) Ascertainment bias in studies of human genome-wide polymorphism. Genome Res 15:1496-1502
Coop G, Przeworski M (2007) An evolutionary view of human recombination. Nat Rev Genet 8:23-34
Evans P D, Gilbert S L, Mekel-Bobrov N, Vallender E J, Anderson J R, Vaez-Azizi L M, Tishkoff S A, Hudson R R, Lahn B T (2005) Microcephalin, a gene regulating brain size, continues to evolve adaptively in humans. Science 309:1717-1720
Funke B, Finn C T, Plocik A M, Lake S, DeRosse P, Kane J M, Kucherlapati R, Malhotra A K (2004) Association of the DTNBP1 locus with schizophrenia in a U.S. population. Am J Hum Genet. 75:891-8
Gibson J, Morton N E, Collins A (2006) Extended tracts of homozygosity in outbred human populations. Hum Mol Genet 15:789-795
Hardy J, Pittman A, Myers A, Fung H C, de Silva R, Duckworth J (2006) Tangle diseases and the tau haplotypes. Alzheimer Dis Assoc Disord 20:60-62
Hirschhorn J N, Daly M J (2005) Genome-wide association studies for common diseases and complex traits. Nat Rev Genet 6:95-108
Hodgkinson C A, Goldman D, Jaeger J, Persaud S, Kane J M, Lipsky R H, Malhotra A K (2004) Disrupted in schizophrenia 1 (DISC 1): association with schizophrenia, schizoaffective disorder, and bipolar disorder. Am J Hum Genet. 75(5):862-72
Kamiya A, Kubo K, Tomoda T, Takaki M, Youn R, Ozeki Y, Sawamura N, Park U, Kudo C, Okawa M, et al. (2005) A schizophrenia-associated mutation of DISCI perturbs cerebral cortex development. Nat Cell Biol 7:1167-1178
Hinds D A, Stuve L L, Nilsen G B, Halperin E, Eskin E, Ballinger D G, Frazer K A, Cox D R (2005) Whole-genome patterns of common DNA variation in three human populations. Science 307:1072-1079
International HapMap Consortium (2005) A haplotype map of the human genome. Nature 437:1299-1320
Kennedy G C, Matsuzaki H, Dong S, Liu W M, Huang J, Liu G, Su X; Cao M, Chen W, Zhang J, et al (2003) Large-scale genotyping of complex DNA. Nat Biotechnol 21:1233-7
Kim Y, Nielsen R (2004) Linkage disequilibrium as a signature of selective sweeps. Genetics 167:1513-1524
Kyosseva S V, Elbein A D, Hutton T L, Griffin S T, Mrak R E, Sturner W Q, Karson C N (2000) Increased levels of transcription factors Elk-1, cyclic adenosine monophosphate response element-binding protein, and activating transcription factor 2 in the cerebellar vermis of schizophrenic patients. Arch Gen Psychiatry 57:685-691
Law A J, Kleinman J E, Weinberger D R, Weickert C S (2007) Disease-associated intronic variants in the ErbB4 gene are related to altered ErbB4 splice-variant expression in the brain in schizophrenia. Hum Mol Genet 16:129-141
Lencz T, Morgan T V, Athanasiou M, Dain B, Reed C R, Kane J M, Kucherlapati R, Malhotra A K (2007) Converging evidence for a pseudoautosomal cytokine receptor gene locus in schizophrenia. Mol Psychiatry 12:572-580
Lewis C M, Levinson D F, Wise L H, DeLisi L E, Straub R E, Hovatta I, Williams N M, Schwab S G, Pulver A E, Faraone S V, et al. (2003) Genome scan meta-analysis of schizophrenia and bipolar disorder, part II: Schizophrenia. Am J Hum Genet 73:34-48
McCarroll S A, Hadnott T N, Perry G H, Sabeti P C, Zody M C, Barrett J C, Dallaire S, Gabriel S B, Lee C, Daly M J, et al. (2006) Common deletion polymorphisms in the human genome. Nat Genet 38:86-92
McVean G A, Myers S R, Hunt S, Deloukas P, Bentley D R, Donnelly P (2004) The fine-scale structure of recombination rate variation in the human genome. Science 304:581-584
Mimics K, Middleton F A, Marquez A, Lewis D A, Levitt P (2000) Molecular characterization of schizophrenia viewed by microarray analysis of gene expression in prefrontal cortex. Neuron 28:53-67
Miyazawa H, Kato M, Awata T, Kohda M, Iwasa H, Koyama N, Tanaka T, Kyo S, Okazaki Y, Hagiwara K (2007) Homozygosity haplotype allows a genomewide search for the autosomal segments shared among patients. Am J Hum Genet 80:1090-1102
Poline J B, Worsley K J, Evans A C, Friston K J (1997) Combining spatial extent and peak intensity to test for activations in functional imaging. Neuroimage 5:83-96
Pritchard J K, Stephens M, Donnelly P (2000) Inference of population structure using multilocus genotype data. Genetics 155:945-59
Redon R, Ishikawa S, Fitch K R, Feuk L, Perry G H, Andrews T D, Fiegler H, Shapero M H, Carson A R, Chen W, et al. (2006) Global variation in copy number in the human genome. Nature 444:444-454
Reich D E, Schaffner S F, Daly M J, McVean G, Mullikin J C, Higgins J M, Richter D J, Lander E S, Altshuler D (2002) Human genome sequence variation and the influence of gene history, mutation and recombination. Nat Genet 32:135-142
Sabeti P C, Reich D E, Higgins J M, Levine H Z, Richter D J, Schaffner S F, Gabriel S B, Platko J V, Patterson N J, McDonald G J, et al. (2002) Detecting recent positive selection in the human genome from haplotype structure. Nature 419:832-837
Sharp A J, Hansen S, Selzer R R, Cheng Z, Regan R, Hurst J A, Stewart H, Price S M, Blair E, Hennekam R C, et al. (2006) Discovery of previously unidentified genomic disorders from the duplication architecture of the human genome. Nat Genet 38:1038-1042
Shriver M D, Parra E J, Dios S, Bonilla C, Norton H, Jovel C, Pfaff C, Jones C, Massac A, Cameron N, et al (2003) Skin pigmentation, biogeographical ancestry and admixture mapping. Hum Genet 112:387-99
Simon-Sanchez J, Scholz S, Fung H C, Matarin M, Hernandez D, Gibbs J R, Britton A, de Vrieze F W, Peckham E, Gwinn-Hardy K, et al. (2007) Genome-wide SNP assay reveals structural genomic variation, extended homozygosity and cell-line induced alterations in normal individuals. Hum Mol Genet 16:1-14
Storey J D, Tibshirani R (2003) Statistical significance for genomewide studies. Proc Natl Acad Sci 100:9440-9445
Williamson S H, Hubisz M J, Clark A G, Payseur B A, Bustamante C D, Nielsen R (2007) Localizing recent adaptive evolution in the human genome. PLoS Genet electronically published Apr. 20, 2007.
Voight B F, Kudaravalli S, Wen X, Pritchard J K (2006) A map of recent positive selection in the human genome. PLoS Biol 4:e72
Wang E T, Kodama G, Baldi P, Moyzis R K (2006) Global landscape of recent inferred Darwinian selection for Homo sapiens. Proc Natl Acad Sci 103:135-140
Xu B, Wratten N, Charych E I, Buyske S, Firestein B L, Brzustowicz LM (2005) Increased expression in dorsolateral prefrontal cortex of CAPON in schizophrenia and bipolar disorder. PLoS Med 2:e263
Zheng Y, Li H, Qin W, Chen W, Duan Y, Xiao Y, Li C, Zhang J, Li X, Feng G, He L (2005) Association of the carboxyl-terminal PDZ ligand of neuronal nitric oxide synthase gene with schizophrenia in the Chinese Han population. Biochem Biophys Res Commun 328:809-15

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1. A method of determining the relative risk of a human subject for manifesting schizophrenia, the method comprising determining the presence of a first run of homozygosity (ROH) in the genome of the subject,
wherein the presence of the first ROH indicates the subject has an increased risk for manifesting schizophrenia over a subject not having the first ROH,
wherein the first ROH is a series of consecutive single nucleotide polymorphism (SNP) positions that are homozygous in the subject from one of roh250, roh321, roh314, roh52, roh15, roh129, roh291, roh55, or roh173 as defined in Table 2.

2. The method of claim 1, wherein the first ROH is a series of at least 50 consecutive homozygous SNP positions.

3. The method of claim 1, wherein the first ROH is a series of at least 100 consecutive homozygous SNP positions.

4. The method of claim 1, wherein the first ROH is a series of all of the SNP positions that are homozygous in the subject from roh250, roh321, roh314, roh52, roh15, roh129, roh291, roh55, or roh173.

5. The method of claim 1, the method further comprising determining the presence of a second ROH in the genome of the subject,
wherein the second ROH is from one of roh250, roh321, roh314, roh52, roh15, roh129, roh291, roh55, or roh173 that is different from the first ROH,
wherein the presence of the second ROH indicates the subject has an increased risk for manifesting schizophrenia over a subject not having the second ROH.

6. The method of claim 1, wherein the presence of roh250 is determined.

7. The method of claim 1, wherein positions in the genome of the subject corresponding to each of roh250, roh321, roh314, roh52, roh15, roh129, roh291, roh55, and roh173 are evaluated for the consecutive homozygous SNP positions, wherein an increasing number of ROHs present in the subject indicates an increasing risk in the subject for manifesting schizophrenia.

8. The method of claim 1, wherein the subject is an embryo or fetus.

9. The method of claim 8, wherein the subject is an embryo.

10. The method of claim 8, wherein the subject is a fetus.

11. A method of determining the relative risk of a human subject for manifesting schizophrenia, the method comprising
determining whether the subject has a run of homozygosity (ROH) that contains at least 80% of the SNPs in at least one of the three locations identified in Supplementary Table 2 as correlated with schizophrenia,
wherein a subject having an ROH that contains at least 80% of the SNPs in at least one of the three locations identified in Supplementary Table 2 has an increased risk for manifesting schizophrenia over a subject not having such an ROH.

12. The method of claim 11, comprising
determining whether the subject has an ROH that contains at least 90% of the SNPs in at least one of the three locations identified in Supplementary Table 2 as correlated with schizophrenia,
wherein a subject having an ROH that contains at least 90% of the SNPs in at least one of the three locations identified in Supplementary Table 2 has an increased risk for manifesting schizophrenia over a subject not having such an ROH.

13. The method of claim 11, comprising
determining whether the subject has an ROH that contains 100% of the SNPs in at least one of the three locations identified in Supplementary Table 2 as correlated with schizophrenia,
wherein a subject having an ROH that contains 100% of the SNPs in at least one of the three locations identified in Supplementary Table 2 has an increased risk for manifesting schizophrenia over a subject not having such an ROH.

14. A method of screening a human embryo in vitro for the risk of becoming a human manifesting schizophrenia, the method comprising determining the presence of a first run of homozygosity (ROH) in the genome of the embryo,
wherein the presence of the first ROH indicates the embryo has an increased risk for manifesting schizophrenia over an embryo not having the first ROH,
wherein the first ROH is a series of consecutive single nucleotide polymorphism (SNP) positions that are homozygous in the subject from one of roh250, roh321, roh314, roh52, roh15, roh129, roh291, roh55, or roh173 as defined in Table 2.

15. The method of claim 14, wherein positions in the genome of the embryo corresponding to each of roh250, roh321, roh314, roh52, roh15, roh129, roh291, roh55, and roh173 are evaluated for the consecutive homozygous SNP positions, wherein an increasing number of ROHs present in the subject indicates an increasing risk in the subject for manifesting schizophrenia.

16. A method of identifying a single nucleotide polymorphism (SNP) variant affecting the risk of a human subject for manifesting schizophrenia, the method comprising
identifying a run of homozygosity (ROH) present more often in a first population of individuals having schizophrenia than in a second population of individuals not having schizophrenia, then
identifying a single nucleotide polymorphism (SNP) within the ROH or within 500 kB of the ROH, where a first variant of the SNP is present in the first population more often than in the second population,
wherein the presence of the first variant of the SNP in a subject indicates that the subject has a greater risk for manifesting schizophrenia than the absence of the first variant,
wherein an ROH is a series of consecutive known SNP positions that are homozygous in the genome of an individual.

17. The method of claim 16, wherein the ROH is one of roh250, roh321, roh314, roh52, roh15, roh129, roh291, roh55, or roh173 as defined in Table 2.

18. The method of claim 16, wherein the SNP is within an open reading frame.

19. The method of claim 18, wherein the open reading frame is in a gene selected from the group consisting of DYNC2H1, PIK3C3, CRHR1, IMP5, MAPT, STH, KIAA1267, LRRC37A, ARL17, LRRC37A2, NSF, WNT3, WNT9B, GOSR2, RPRML, CDC27, CHN1, ATF2, ATP5GS3, DUSP12, ATF6, OLFML2B, NOS1AP, SGCD, MRPL22, GPHN, C14orf54, MPP5, ATP6V1D, EIF2S1, PLEK2, GULP1, DIRC1, COL3A1, COL5A2, WDR75, SLC40A1, NS3TP1, ASNSD1, ANKAR, OSGEPL1, ORMDL1, PMS1, GDF8, and IMPAD1.

20. A method of determining the relative risk of a human subject for manifesting schizophrenia, the method comprising determining whether the subject has a SNP genotype associated with schizophrenia as identified by the method of claim 16, wherein a subject with the SNP genotype has an increased risk for manifesting schizophrenia over a subject with a different genotype.

21. A method of screening for a compound that may affect schizophrenia, the method comprising determining whether the compound affects expression or activity of a gene selected from the group consisting of DYNC2H1, CRHR1, IMP5, MAPT, STH, KIAA1267, LRRC37A, ARL17, LRRC37A2, WNT3, WNT9B, GOSR2, RPRML, CDC27, CHN1, ATP5GS3, DUSP12, ATF6, OLFML2B, SGCD, MRPL22, GPHN, C14orf54, MPP5, ATP6V1D, EIF2S1, PLEK2, GULP1, DIRC1, COL3A1, COL5A2, WDR75, SLC40A1, NS3TP1, ASNSD1, ANKAR, OSGEPL1, ORMDL1, PMS1, GDF8, IMPAD1, SNTG1 and SORCS1,
wherein a compound that affects expression or activity of the gene may affect schizophrenia.

22. The method of claim 21, wherein the gene is MAPT, GPHN, SNTG1 or SORCS1.

23. The method of claim 21, wherein the compound is contacted with a product of the gene then the activity of the gene product is measured.

24. The method of claim 23, wherein the compound is contacted with the product of the gene in vitro.

25. The method of claim 23, wherein the compound is contacted with a cell that expresses the product of the gene such that the compound contacts the product of the gene.

26. The method of claim 21, wherein the compound is contacted with a cell that is capable of expressing the gene, and expression of the gene is measured and compared to expression of the gene in a cell that is not contacted with the compound.

27. The method of claim 21, wherein the compound is administered to a mammal and activity of a product of the gene is measured and compared to activity of the product of the gene in a mammal that is not administered the compound.

28. The method of claim 21, wherein the compound is administered to a mammal and expression of the gene is measured and compared to expression of the gene in a mammal that is not administered the compound.
